(19) United States
(12) Patent Application Publication
(10) Pub. No.: US 2019/0150910 A1
(43) Pub. Date: May 23, 2019

(54) Bone Anchors and Screws

(71) Applicant: HD LifeSciences LLC, Woburn, MA (US)

(72) Inventors: Christopher L. Jones, Bellingham, MA (US); Ian Helmar, Beverly, MA (US); Lucas Diehl, Beverly, MA (US); John F. Sullivan, Pelham, NH (US); Christine Emery, Somerville, MA (US); Jason Tinley, Fort Worth, TX (US); Kevin Chappuis, Malden, MA (US)

(21) Appl. No.: 16/251,383

(22) Filed: Jan. 18, 2019

Related U.S. Application Data
(63) Continuation-in-part of application No. 15/876,695, filed on Jan. 22, 2018.
Application No. 15/876,695 is a continuation-in-part of application No. 15/941,193, filed on Mar. 30, 2018.
(60) Provisional application No. 62/480,383, filed on Apr. 1, 2017.
(60) Provisional application No. 62/480,393, filed on Apr. 1, 2017.
(60) Provisional application No. 62/619,260, filed on Jan. 19, 2018.
(60) Provisional application No. 62/480,383, filed on Apr. 1, 2017.
(60) Provisional application No. 62/480,393, filed on Apr. 1, 2017.
(60) Provisional application No. 62/619,260, filed on Jan. 19, 2018.
(60) Provisional application No. 62/480,388, filed on Apr. 1, 2017.
(60) Provisional application No. 62/619,260, filed on Jan. 19, 2018.

(51) Int. Cl. A61B 17/04; A61F 2/30; A61B 17/86; A61F 2/28

(52) U.S. Cl. CPC A61B 17/0401; A61F 2/3094; A61B 17/86; A61F 2/28; A61F 2/30771; A61F 2002/3097; A61B 2017/0412; A61F 2220/0025; A61F 2310/00023; A61F 2002/30841; A61F 2002/30968; A61F 2230/0069

(57) ABSTRACT

The variable or adjustable depth medical implants disclosed herein are cable of depth adjustment prior to implantation. The variable depth implants permit a single implant to provide multiple footprint configurations, allowing a surgeon adjustability in the operating room. The implants can comprise a metallic lattice designed for specific physical properties, such as an elastic modulus. In some examples, the main body of the implant is taller than the adjustable portion of the implant so that the physical properties of the main body of the implant are controlling at the implant site. In some embodiments, the variable implant is constructed in an additive process as a single unit.

Disclosed herein is, in some embodiments, a multi-segment bone anchor configured to allow variable bone ingrowth or attachment between each segment. In some versions, a lower segment is configured to allow bone ingrowth and a detachable upper segment is configured to prevent bone ingrowth, making the bulk of the bone anchor removable.

### CROSS REFERENCE TO RELATED APPLICATIONS

This application is a continuation-in-part application of U.S. Nonprovisional patent application Ser. No. 15/876,695 filed Jan. 22, 2018, which claims the benefit of U.S. Provisional Patent Application No. 62/480,383 filed Apr. 1, 2017, U.S. Provisional Patent Application No. 62/480,393 filed Apr. 1, 2017, and U.S. Provisional Patent Application No. 62/619,260 filed Jan. 19, 2018, which are hereby incorporated by reference in their entirety; and a continuation-in-part application of U.S. Nonprovisional patent application Ser. No. 15/941,193 filed Mar. 30, 2018, which claims the benefit of U.S. Provisional Patent Application No. 62/480,388 filed Apr. 1, 2017, U.S. Provisional Patent Application No. 62/480,383 filed Apr. 1, 2017, U.S. Provisional Patent Application No. 62/480,393 filed Apr. 1, 2017, and U.S. Provisional Patent Application No. 62/619,260 filed Jan. 19, 2018, which are hereby incorporated by reference in their entirety. This application also claims the benefit of U.S. Provisional Patent Application No. 62/619,260 filed Jan. 19, 2018, which is hereby incorporated by reference in its entirety.

### FIELD OF THE INVENTION

The present invention relates to medical implants, in particular, to medical implants configured to permanently or temporarily attach to bone.

### BACKGROUND OF THE INVENTION

Medical implants, including bone anchors and screws, can be constructed using a wide range of materials, including metallic materials, Polyether ether ketone (hereinafter “PEEK”), ceramic materials and various other materials or composites thereof. There are competing priorities when selecting a material for an implant in order for the implant to pass regulatory testing. Some priorities when designing an implant could include strength, stiffness, fatigue resistance and radiolucency, and often some compromise is made during the design process.

### BRIEF SUMMARY OF THE INVENTION

The present invention, in some aspects, provides an anchoring implant configured for fixation to bone. In some embodiments, the bone anchor comprises multiple segments substantially aligned axially, each with a different volumetric density. In other embodiments, the bone anchor comprises multiple segments substantially aligned axially, each comprising different materials. In other embodiments, the bone anchor comprises multiple segments substantially aligned axially and configured to promote bone ingrowth in one segment and configured to prevent bone ingrowth in another segment.

In one embodiment, the bone anchor can comprise of an upper portion and lower portion capable of selectable fixation to one another in the axial direction. The upper portion may comprise a material that is nonresorbable to allow for later removal from the body. The lower portion may comprise a resorbable material or a lattice structure that allows for bone ingrowth to provide permanent fixation to adjacent bone or tissue. The mechanism between the upper portion and lower portion preferably allows secure attachment and is capable of being detached after implantation. The connecting mechanism can comprise various appropriate fasteners, including but not limited to, correspondingly threaded fasteners, pins, an interference fit, clips or snaps.

### BRIEF DESCRIPTION OF THE SEVERAL VIEWS OF THE DRAWINGS

FIG. 1 is an isometric view of a single modified rhombic dodecahedron unit cell containing a full modified rhombic dodecahedron structure along with radial struts that comprise portions of adjacent unit cells.

FIG. 2 is a side view of a single modified rhombic dodecahedron unit cell showing the configuration of interconnections when viewed from a lateral direction.

FIG. 3 is a side view of a single modified rhombic dodecahedron unit cell where the central void is being measured using the longest dimension method.

FIG. 4 is a side view of a single modified rhombic dodecahedron unit cell where an interconnection is being measured using the longest dimension method.

FIG. 5 is a side view of the central void of a modified rhombic dodecahedron unit cell being measured with the largest sphere method.

FIG. 6 is a view from a direction normal to the planar direction of an interconnection being measured with the largest sphere method.

FIG. 7 is an isometric view of a single radial dodeca-rhombus unit cell.

FIG. 8 is a side view of a single radial dodeca-rhombus unit cell.

FIG. 9 is an isometric view of an example of a single node and single strut combination that could be used in a radial dodeca-rhombus unit cell.

FIG. 10 is a side view of an example of a single node and single strut combination that could be used in a radial dodeca-rhombus unit cell.

FIG. 11 is a side view of a single node and single strut combination configured for use in a lattice with an elastic modulus of approximately 3 GPa, viewed from the corner of the volume defining the bounds of the combination.

FIG. 12 is a side view of a single node and single strut combination configured for use in a lattice with an elastic modulus of approximately 4 GPa, viewed from the corner of the volume defining the bounds of the combination.

FIG. 13 is a side view of a single node and single strut combination configured for use in a lattice with an elastic modulus of approximately 10 GPa, viewed from the corner of the volume defining the bounds of the combination.

FIG. 14 is a side view of a single node and two adjacent struts viewed from the corner of the volume defining the bounds of the combination and the lateral separation angle.

FIG. 15 is an isometric view of a sub-unit cell comprised of a single node and four struts.

FIG. 16 is an isometric view of two sub-unit cells in a stacked formation where the upper sub-unit cell is inverted and fixed to the top of the lower sub-unit cell.

FIG. 17 is an isometric view of eight sub-unit cells stacked together to form a single unit cell.

FIG. 18A is a side view of a first exemplary upper bone anchor configured to receive a detachable lower bone anchor.

FIG. 18B is a side view of a first exemplary detachable lower bone anchor.

FIG. 18C is a side view of an alternative first exemplary detachable lower bone anchor.

FIG. 19A is a side view of a second exemplary upper bone anchor configured to receive a detachable lower bone anchor.

FIG. 19B is a side view of a second exemplary detachable lower bone anchor.

FIG. 20A is a side view of a third exemplary upper bone anchor configured to receive a detachable lower bone anchor.

FIG. 20B is a side view of a third exemplary detachable lower bone anchor.

FIG. 20C is a side view of an alternative third exemplary detachable lower bone anchor.

FIG. 21A is a top sectioned view of a fourth exemplary detachable lower bone anchor.

FIG. 21B is a top sectioned view of a fifth exemplary detachable lower bone anchor.

FIG. 22 is a side view of an example of a bone anchor assembly with an upper bone anchor and a lower bone anchor.

FIG. 23 is a side sectioned view of a detachable lower bone anchor that has been implanted in bone and after the upper bone anchor has been removed.

FIG. 24 is a side view of an example of a bone anchor assembly with an upper bone anchor, a lower bone anchor and a cannula.

FIG. 25 is a side sectioned view of a detachable lower bone anchor with a cannula that has been implanted in bone and after the upper bone anchor has been removed.

FIG. 26 is a side view of an example of a bone anchor assembly with an upper bone anchor, a lower bone anchor and a cannula.

FIG. 27 is a side sectioned view of a detachable lower bone anchor with a cannula that has been implanted in bone and after the upper bone anchor has been removed.

### DETAILED DESCRIPTION OF THE INVENTION

In many situations, it is desirable to use an implant that is capable of bone attachment or osteointegration over time. It is also desirable in many situations to use an implant that is capable of attachment or integration with living tissue. Examples of implants where attachment to bone or osteointegration is beneficial include, but are not limited to, cervical, lumbar, and thoracic interbody fusion implants, vertebral body replacements, osteotomy wedges, dental implants, bone stems, acetabular cups, cranio-facial plating, bone replacement and fracture plating. In many applications, it is also desirable to stress new bone growth to increase its strength. According to Wolff's law, bone will adapt to stresses placed on it so that bone under stress will grow stronger and bone that isn't stressed will become weaker.

In some aspects, the systems and methods described herein can be directed toward implants that are configured for osteointegration and stimulating adequately stressed new bone growth. Many of the exemplary implants of the present invention are particularly useful for use in situations where it is desirable to have strong bone attachment and/or bone growth throughout the body of an implant. Whether bone growth is desired only for attachment or throughout an implant, the present invention incorporates a unique lattice structure that can provide mechanical spacing, a scaffold to support new bone growth and a modulus of elasticity that allows new bone growth to be loaded with physiological forces. As a result, the present invention provides implants that grow stronger and healthier bone for more secure attachment and/or for a stronger bone after the implant osteointegrates.

The exemplary embodiments of the invention presented can be comprised, in whole or in part, of a lattice. A lattice, as used herein, refers to a three-dimensional material with one or more interconnected openings that allow a fluid to communicate from one location to another location through an opening. A three-dimensional material refers to a material that fills a three-dimensional space (i.e., has height, width and length). Lattices can be constructed by many means, including repeating various geometric shapes or repeating random shapes to accomplish a material with interconnected openings. A lattice can alternatively be constructed by drilling holes in two directions of a material. For example, a square lattice can be constructed by generating square holes in one direction, such as a lateral direction, and by generating round holes in another direction, such as the caudal-rostral direction. An opening in a lattice is any area within the bounds of the three-dimensional material that is devoid of that material. Therefore, within the three-dimensional boundaries of a lattice, there is a volume of material and a volume that is devoid of that material.

The material that provides the structure of the lattice is referred to as the primary material. The structure of a lattice does not need to provide structural support for any purpose, but rather refers to the configuration of the openings and interconnections that comprise the lattice. An opening in a lattice may be empty, filled with a gaseous fluid, filled with a liquid fluid, filled with a solid or partially filled with a fluid and/or solid. Interconnections, with respect to openings, refer to areas devoid of the primary material and that link at least two locations together. Interconnections may be configured to allow a fluid to pass from one location to another location.

A lattice can be defined by its volumetric density, meaning the ratio between the volume of the primary material and the volume of voids presented as a percentage for a given three-dimensional material. The volume of voids is the difference between the volume of the bounds of the three-dimensional material and the volume of the primary material. The volume of voids can comprise of the volume of the openings, the volume of the interconnections and/or the volume of another material present. For example, a lattice with a 30% volumetric density would be comprised of 30% primary material by volume and 70% voids by volume over a certain volume. A lattice with a 90% volumetric density would be comprised of 90% primary material by volume and 10% voids by volume over a certain volume. In three-dimensional materials with a volumetric density of less than 50%, the volume of the primary material is less than the volume of voids. While the volumetric density refers to the volume of voids, the voids do not need to remain void and can be filled, in whole or in part, with a fluid or solid prior to, during or after implantation.

Lattices comprising repeating geometric patterns can be described using the characteristics of a repeating unit cell. A unit cell in a repeating geometric lattice is a three-dimensional shape capable of being repeated to form a lattice. A repeating unit cell can refer to multiple identical unit cells that are repeated over a lattice structure or a pattern through all or a portion of a lattice structure. Each unit cell is comprised of a certain volume of primary material and a certain void volume, or in other words, a spot volumetric density. The spot volumetric density may cover as few as a partial unit cell or a plurality of unit cells. In many situations, the spot volumetric density will be consistent with the material's volumetric density, but there are situations where it could be desirable to locally increase or decrease the spot volumetric density.

Unit cells can be constructed in numerous volumetric shapes containing various types of structures. Unit cells can be bound by a defined volume of space to constrict the size of the lattice structure or other type of structure within the unit cell. In some embodiments, unit cells can be bound by volumetric shapes, including but not limited to, a cubic volume, a cuboid volume, a hexahedron volume or an amorphous volume. The unit cell volume of space can be defined based on a number of faces that meet at corners. In examples where the unit cell volume is a cubic, cuboid or hexahedron volume, the unit cell volume can have six faces and eight corners, where the corners are defined by the location where three faces meet. Unit cells may be interconnected in some or all areas, not interconnected in some or all areas, of a uniform size in some or all areas or of a nonuniform size in some or all areas. In some embodiments disclosed herein that use a repeating geometric pattern, the unit cells can be defined by a number of struts defining the edges of the unit cell and joined at nodes about the unit cell. Unit cells so defined can share certain struts among more than one unit cell, so that two adjacent unit cells may share a common planar wall defined by struts common to both cells. In some embodiments disclosed herein that use a repeating geometric pattern, the unit cells can be defined by a node and a number of struts extending radially from that node.

While the present application uses volumetric density to describe exemplary embodiments, it is also possible to describe them using other metrics, including but not limited to cell size, strut size or stiffness. Cell size may be defined using multiple methods, including but not limited to cell diameter, cell width, cell height and cell volume. Strut size may be defined using multiple methods, including but not limited to strut length and strut diameter.

Repeating geometric patterns are beneficial for use in lattice structures contained in implants because they can provide predictable characteristics. Many repeating geometric shapes may be used as the unit cell of a lattice, including but are not limited to, rhombic dodecahedron, diamond, dodecahedron, square, pentagonal, hexagonal, octagonal, sctet struts, trunic octa, diagonal struts, other known geometric structures, and rounded, reinforced, weakened, or simplified versions of each geometry.

Lattices may also be included in implants as a structural component or a nonstructural component. Lattices used in structural applications may be referred to herein as structural lattices, load-bearing lattices or stressed lattices. In some instances, structural lattices, load-bearing lattices or stressed lattices may be simply referred to as a lattice. Repeating geometric shaped unit cells, particularly the rhombic dodecahedron, are well suited, in theory, for use in structural lattices because of their strength to weight ratio. To increase the actual strength and fatigue resistance of a rhombic dodecahedron lattice, the present invention, in some embodiments, includes a modified strut comprised of triangular segments, rather than using a strut with a rectangular or circular cross section. Some embodiments herein also modify the angles defining the rhombic faces of a rhombic dodecahedron to change the lattice's elastic modulus and fatigue resistance. The use of triangular segments provides a lattice with highly predictable printed properties that approach the theoretical strength values for a rhombic dodecahedron lattice.

In structural lattice applications, the strength and elastic modulus of the lattice can be approximated by the volumetric density. When the volumetric density increases, the strength and the elastic modulus increases. Compared to other porous structures, the lattice of the present invention has a higher strength and elastic modulus for a given volumetric density because of its ability to use the high strength to weight benefits of a rhombic dodecahedron, modified rhombic dodecahedron or radial dodeca-rhombus unit cell.

When configured to provide support for bone or tissue growth, a lattice may be referred to as a scaffold. Lattices can be configured to support bone or tissue growth by controlling the size of the openings and interconnections disposed within the three-dimensional material. A scaffold, if used on the surface of an implant, may provide an osteointegration surface that allows adjacent bone to attach to the implant. A scaffold may also be configured to provide a path that allows bone to grow further than a mere surface attachment. Scaffolds intended for surface attachment are referred to herein as surface scaffolds. A surface scaffold may be one or more unit cells deep, but does not extend throughout the volume of an implant. Scaffolds intended to support in-growth beyond mere surface attachment are referred to herein as bulk scaffolds. Scaffolds may also be included in implants as a structural component or a nonstructural component. Scaffolds used in structural applications may be referred to herein as structural scaffolds, load-bearing scaffolds or stressed scaffolds. In some instances, structural scaffolds, load-bearing scaffolds or stressed scaffolds may be simply referred to as a scaffold. In some instances, the use of the term scaffold may refer to a material configured to provide support for bone or tissue growth, where the material is not a lattice.

The scaffolds described herein can be used to promote the attachment or in-growth of various types of tissue found in living beings. As noted earlier, some embodiments of the scaffold are configured to promote bone attachment and in-growth. The scaffolds can also be configured to promote attachment of in-growth of other areas of tissue, such as fibrous tissue. In some embodiments, the scaffold can be configured to promote the attachment or in-growth of multiple types of tissue. Some embodiments of the scaffolds are configured to be implanted near or abutting living tissue. Near living tissue includes situations where other layers, materials or coatings are located between a scaffold and any living tissue.

In some embodiments, the present invention uses bulk scaffolds with openings and interconnections that are larger than those known in the art. Osteons can range in diameter from about 100 μm and it is theorized that a bundle of osteons would provide the strongest form of new bone growth. Bone is considered fully solid when it has a diameter of greater than 3 mm so it is theorized that a bundle of osteons with a diameter equaling approximately half of that value would provide significant strength when grown within a scaffold. It is also theorized that osteons may grow in irregular shapes so that the cross-sectional area of an osteon could predict its strength. A cylindrical osteon growth with a 3 mm diameter has a cross-sectional area of approximately 7 square mm and a cylindrical osteon with a 1.5 mm diameter has a cross-sectional area of 1.8 square mm. It is theorized that an osteon of an irregular shape with a cross-sectional area of at least 1.8 square millimeters could provide a significant strength advantage when grown in a scaffold.

Most skilled in the art would indicate that pores or openings with a diameter or width between 300 μm to 900 μm, with a pore side of 600 μm being ideal, provide the best scaffold for bone growth. Instead, some embodiments of the present invention include openings and interconnections with a diameter or width on the order of 1.0 to 15.0 times the known range, with the known range being 300 μm to 900 μm, resulting in openings from 0.07 mm² up to 145 mm² cross sectional area for bone growth. In some examples, pores or openings with a diameter or width between and including 100 μm to 300 μm could be beneficial. Some examples include openings and interconnections with a diameter on the order of 1.0 to 5.0 times the known range. It has been at least theorized that the use of much larger openings and interconnections than those known in the art will allow full osteons and solid bone tissue to form throughout the bulk scaffold, allowing the vascularization of new, loadable bone growth. In some examples, these pores may be 3 mm in diameter or approximately 7 mm² in cross sectional area. In other examples, the pores are approximately 1.5 mm in diameter or approximately 1.75 mm² in cross sectional area. The use of only the smaller diameter openings and interconnections known in the art are theorized to limit the penetration of new bone growth into a bulk scaffold because the smaller diameter openings restrict the ability of vascularization throughout the bulk scaffold.

A related structure to a lattice is a closed cell material. A closed cell material is similar to a lattice, in that it has openings contained within the bounds of a three-dimensional material, however, closed cell materials generally lack interconnections between locations through openings or other pores. A closed cell structure may be accomplished using multiple methods, including the filling of certain cells or through the use of solid walls between the struts of unit cells. A closed cell structure can also be referred to as a cellular structure. It is possible to have a material that is a lattice in one portion and a closed cell material in another. It is also possible to have a closed cell material that is a lattice with respect to only certain interconnections between openings or vice versa. While the focus of the present disclosure is on lattices, the structures and methods disclosed herein can be easily adapted for use on closed cell structures within the inventive concept.

The lattice used in the present invention can be produced from a range of materials and processes. When used as a scaffold for bone growth, it is desirable for the lattice to be made of a biocompatible material that allows for bone attachment, either to the material directly or through the application of a bioactive surface treatment. In one example, the scaffold is comprised of an implantable metal. Implantable metals include, but are not limited to, zirconium, stainless steel (316 & 316L), tantalum, nitinol, cobalt chromium alloys, bulk metallic glass, titanium and tungsten, and alloys thereof. Scaffolds comprised of an implantable metal may be produced using an additive metal fabrication or 3D printing process. Appropriate production processes include, but are not limited to, direct metal laser sintering, selective laser sintering, selective laser melting, electron beam melting, laminated object manufacturing, infiltrated binder jetting, print-to-cast, print-to-plating, bulk metallic glass extrusion, nano-laser printing and directed energy deposition.

In another example, the lattice of the present invention is comprised of an implantable metal with a bioactive coating. Bioactive coatings include, but are not limited to, coatings to accelerate bone growth, anti-thrombogenic coatings, anti-microbial coatings, hydrophobic or hydrophilic coatings, and hemophobic, superhemophobic, or hemophilic coatings. Coatings that accelerate bone growth include, but are not limited to, calcium phosphate, hydroxyapatite (“HA”), silicate glass, stem cell derivatives, bone morphogenic proteins, titanium plasma spray, titanium beads and titanium mesh. Anti-thrombogenic coatings include, but are not limited to, low molecular weight fluoro-oligomers. Anti-microbial coatings include, but are not limited to, silver, organosilane compounds, iodine and silicon-nitride. Superhemophobic coatings include fluorinated nanotubes.

In another example, the lattice is made from a titanium alloy with an optional bioactive coating. In particular, Ti6Al4V ELI wrought (American Society for Testing and Materials (“ASTM”) F136) is a particularly well-suited titanium alloy for scaffolds. While Ti6Al4V ELI wrought is the industry standard titanium alloy used for medical purposes, other titanium alloys, including but not limited to, unalloyed titanium (ASTM F67), Ti6Al4V standard grade (ASTM F1472), Ti6Al7Nb wrought (ASTM 1295), Ti5Al2.5Fe wrought (British Standards Association/International Standard Organization Part 10), CP and Ti6Al4V standard grade powders (ASTM F1580), Ti13Nb13Zr wrought (ASTM F1713), the lower modulus Ti-24Nb-4Zr-8Sn and Ti12Mo6Zr2Fe wrought (ASTM F1813) can be appropriate for various embodiments of the present invention.

Titanium alloys are an appropriate material for scaffolds because they are biocompatible and allow for bone attachment. Various surface treatments can be done to titanium alloys to increase or decrease the level of bone attachment. Bone will attach to even polished titanium, but titanium with a surface texture allows for greater bone attachment. Methods of increasing bone attachment to titanium may be produced through a forging or milling process, sandblasting, acid etching, and the use of a bioactive coating. Titanium parts produced with an additive metal fabrication or 3D printing process, such as direct metal laser sintering, can be treated with an acid bath to reduce surface stress risers, normalize surface topography, and improve surface oxide layer, while maintaining surface roughness and porosity to promote bone attachment.

Additionally, Titanium or other alloys may be treated with heparin, heparin sulfate (HS), glycosaminoglycans (GAG), chondroitin-4-sulphate (C4S), chondroitin-6-sulphate (C6S), hyaluronan (HY), bone marrow aspirate (BMA), and other proteoglycans with or without an aqueous calcium solution. Such treatment may occur while the material is in its pre-manufacturing form (often powder) or subsequent to manufacture of the structure.

While a range of structures, materials, surface treatments and coatings have been described, it is believed that a lattice using a repeating modified rhombic dodecahedron (hereinafter “MRDD”) unit cell can present a preferable combination of stiffness, strength, fatigue resistance, and conditions for bone ingrowth. In some embodiments, the repeating MRDD lattice comprises titanium or a titanium alloy. A generic rhombic dodecahedron (hereinafter “RDD”), by definition, has twelve sides in the shape of rhombuses. When repeated in a lattice, an RDD unit cell is comprised of twenty-four struts that meet at fourteen vertices. The twenty-four struts define the twelve planar faces of the structure. An opening or interconnection is disposed at the center of each planar face, allowing communication from inside the unit cell to outside the unit cell.

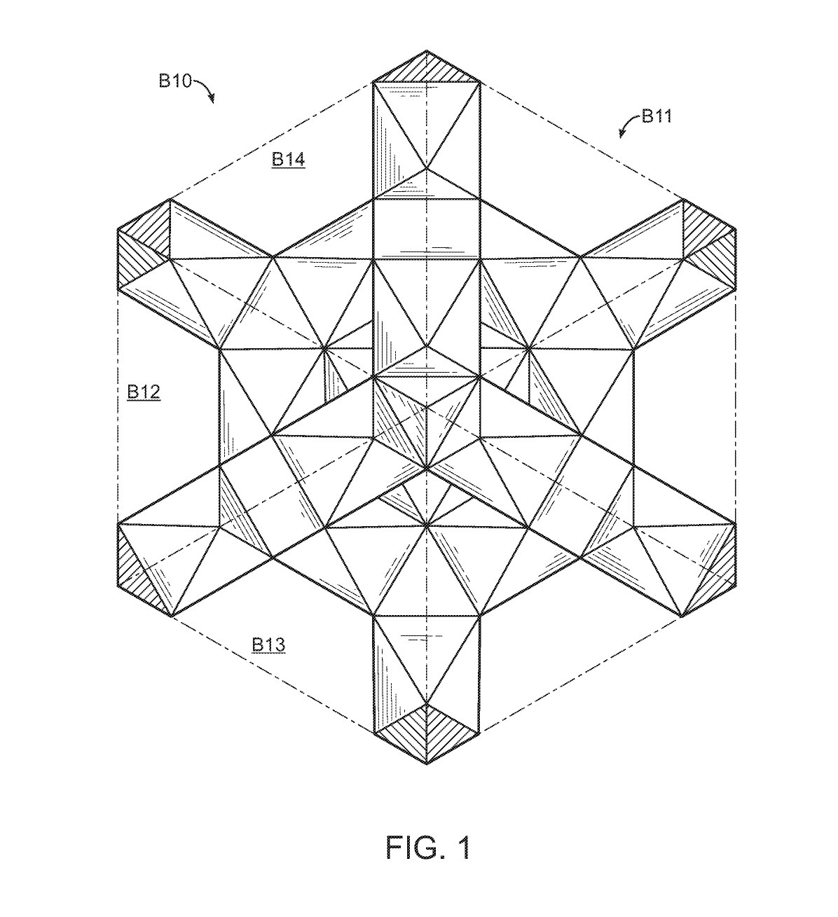

An example of the MRDD unit cell 10 used in the present invention is shown in FIGS. 1-5. FIG. 1 illustrates an isometric view of a single MRDD unit cell 10 containing a full MRDD structure along with radial struts 31 that comprise portions of adjacent unit cells. In FIG. 2 is a side view of a single MRDD unit cell 10 showing the configuration of interconnections when viewed from a lateral direction. A top or bottom view of the MRDD unit cell 10 would be substantially the same as the side view depicted in FIG. 2. The MRDD unit cell 10 differs in both structural characteristics and method of design from generic RDD shapes. A generic RDD is comprised of twelve faces, where each face is an identical rhombus with an acute angle of 70.5 degrees and an obtuse angle of 109.5 degrees. The shape of the rhombus faces in a generic RDD do not change if the size of the unit cell or the diameter of the struts are changed because the struts are indexed based on their axis and each strut axis passes through the center of the fourteen nodes or vertices.

In some embodiments of the MRDD, each node 30 is contained within a fixed volume that defines its bounds and provides a fixed point in space for the distal ends of the struts 31. The fixed volume containing the MRDD or a sub-unit cell of the MRDD can comprise of various shapes, including but not limited to, a cubic, cuboid, hexahedron or amorphous volume. Some examples use a fixed volume with six faces and eight corners defined by locations where three faces meet. The orientation of the struts 31 can be based on the center of a node face at its proximate end and the nearest corner of the volume to that node face on its distal end. Each node 30 is preferably an octahedron, more specifically a square bipyramid (i.e., a pyramid and inverted pyramid joined on a horizontal plane). Each node 30, when centrally located in a cuboid volume, more preferably comprises a square plane parallel to a face of the cuboid volume and six vertices. Each node 30 is oriented so that each of the six vertices are positioned at their closest possible location to each of the six faces of the cuboid volume. As used herein, the term “centrally located,” with regards to the node's location within a volume refers to positioning the node at a location substantially equidistant from opposing walls of the volume. In some embodiments, the node 30 can have a volumetric density of 100% and in other embodiments, the node can have a volumetric density of less than 100%. Each face of the square bipyramid node 30 can be triangular and each face can provide a connection point for a strut 31.

The struts 31 can also be octahedrons, comprising an elongate portion of six substantially similar elongate faces and two end faces. The elongate faces can be isosceles triangles with a first internal angle, angle A, and a second internal angle, angle B, where angle B is greater than angle A. The end faces can be substantially similar isosceles triangles to one another with a first internal angle, angle C, and a second internal angle, angle D, where angle D is greater than angle C. Preferably, angle C is greater than angle A.

The strut direction of each strut is a line or vector defining the orientation of a strut and it can be orthogonal or non-orthogonal relative to the planar surface of each node face. In the MRDD and radial dodeca-rhombus structures disclosed herein, the strut direction can be determined using a line extending between the center of the strut end faces, the center of mass along the strut or an external edge or face of the elongate portion of the strut. When defining a strut direction using a line extending between the center of the strut end faces, the line is generally parallel to the bottom face or edge of the strut. When defining a strut direction using a line extending along the center of mass of the strut, the line can be nonparallel to the bottom face or edge of the strut. The octahedron nodes of the MRDD can be scaled to increase or decrease volumetric density by changing the origin point and size of the struts. The distal ends of the struts, however, are locked at the fixed volume corners formed about each node so that their angle relative to each node face changes as the volumetric density changes. Even as the volumetric density of an MRDD unit cell changes, the dimensions of the fixed volume formed about each node does not change. In FIG. 1, dashed lines are drawn between the corners of the MRDD unit cell 10 to show the cube 11 that defines its bounds. In the MRDD unit cell in FIG. 1, the height 12, width 13 and depth 14 of the unit cell are substantially the same, making the area defined by the cube 11.

In some embodiments, the strut direction of a strut 31 can intersect the center of the node and the corner of the cuboid volume nearest to the node face where the strut 31 is fixed. In some embodiments, the strut direction of a strut 31 can intersect just the corner of the cuboid volume nearest to the node face where the strut is fixed. In some embodiments, a reference plane defined by a cuboid or hexahedron face is used to describe the strut direction of a strut. When the strut direction of a strut is defined based on a reference plane, it can be between 0 degrees and 90 degrees from the reference plane. When the strut direction of a strut is defined based on a reference plane, it is preferably eight degrees to 30 degrees from the reference plane.

By indexing the strut orientation to a variable node face on one end and a fixed point on its distal end, the resulting MRDD unit cell can allow rhombus shaped faces with a smaller acute angle and larger obtuse angle than a generic RDD. The rhombus shaped faces of the MRDD can have two substantially similar opposing acute angles and two substantially similar opposing obtuse angles. In some embodiments, the acute angles are less than 70.5 degrees and the obtuse angles are greater than 109.5 degrees. In some embodiments, the acute angles are between 0 degrees and 55 degrees and the obtuse angles are between 125 degrees and 180 degrees. In some embodiments, the acute angles are between 8 degrees and 60 degrees and the obtuse angles are between 120 degrees and 172 degrees. The reduction in the acute angles increases fatigue resistance for loads oriented across the obtuse angle corner to far obtuse angle corner. The reduction in the acute angles and increase in obtuse angles also orients the struts to increase the MRDD's strength in shear and increases the fatigue resistance. By changing the rhombus corner angles from a generic RDD, shear loads pass substantially in the axial direction of some struts, increasing the shear strength. Changing the rhombus corner angles from a generic RDD also reduces overall deflection caused by compressive loads, increasing the fatigue strength by resisting deflection under load.

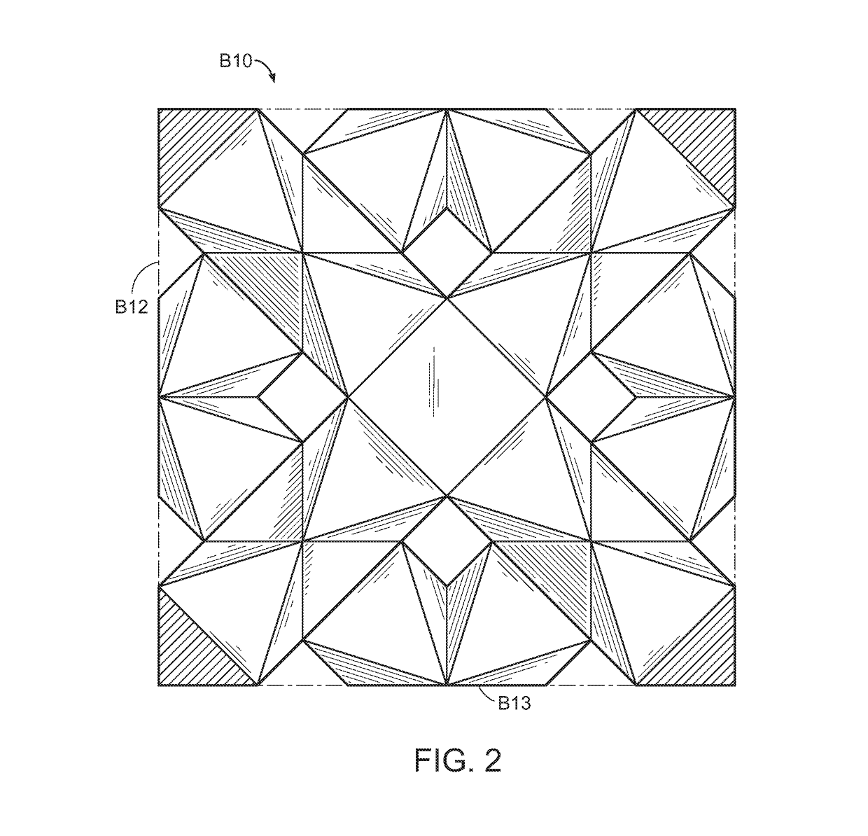

When placed towards the center of a lattice structure, the twelve interconnections of a unit cell 30 connect to twelve different adjacent unit cells, providing continuous paths through the lattice. The size of the central void and interconnections in the MRDD may be defined using the longest dimension method as described herein. Using the longest dimension method, the central void can be defined by taking a measurement of the longest dimension as demonstrated in FIG. 3. In FIG. 3, the longest dimension is labeled as distance AA. The distance AA can be taken in the vertical or horizontal directions (where the directions reference the directions on the page) and would be substantially the same in this example. The interconnections may be defined by their longest measurement when viewed from a side, top or bottom of a unit cell. In FIG. 4, the longest dimension is labeled as distance AB. The distance AB can be taken in the vertical or horizontal directions (where the directions reference the directions on the page). The view in FIG. 4 is a lateral view, however, in this example the unit cell will appear substantially the same when viewed from the top or bottom.

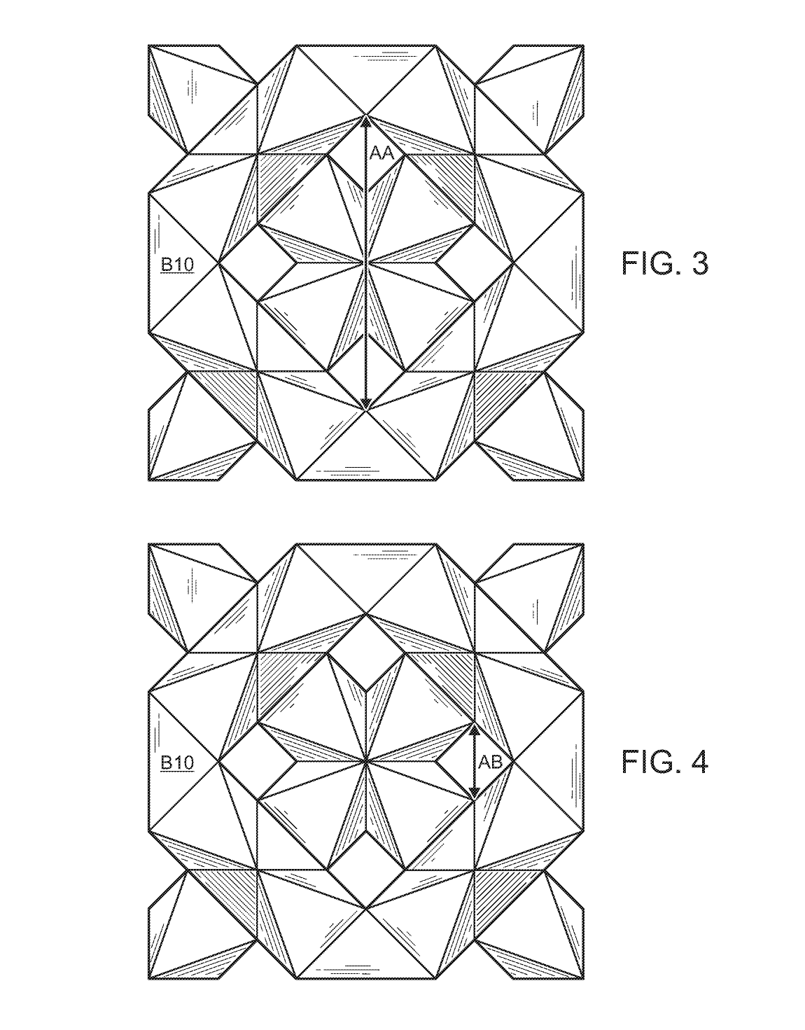

The size of the central void and interconnections can alternatively be defined by the largest sphere method as described herein. Using the largest sphere method, the central void can be defined by the diameter of the largest sphere that can fit within the central void without intersecting the struts. FIG. 5 depicts an example of the largest sphere method being used to define the size of a central void with a sphere with a diameter of BA. FIG. 6 is a view from a direction normal to the planar direction of an interconnection being measured with the largest sphere method. The interconnections are generally rhombus shaped and their size can alternatively be defined by the size of the length and width of three circles drawn within the opening. As shown in FIG. 6, within the plane defining a side, a first circle BB1 is drawn at the center of the opening so that it is the largest diameter circle that can fit without intersecting the struts. A second circle BB2 and third circle BB3 is then drawn so that they are tangential to the first circle BB1 and the largest diameter circles that can fit without intersecting the struts. The diameter of the first circle BB1 is the width of the interconnection and the sum of the diameters of all three circles BB1, BB2 & BB3 represents the length of the interconnection. Using this method of measurement removes the acute corners of the rhombus shaped opening from the size determination. In some instances, it is beneficial to remove the acute corners of the rhombus shaped opening from the calculated size of the interconnections because of the limitations of additive manufacturing processes. For example, if an SLS machine has a resolution of 12 μm where the accuracy is within 5 μm, it is possible that the acute corner could be rounded by the SLS machine, making it unavailable for bone ingrowth. When designing lattices for manufacture on less precise additive process equipment, it can be helpful to use this measuring system to better approximate the size of the interconnections.

Using the alternative measuring method, in some examples, the width of the interconnections is approximately 600 μm and the length of the interconnections is approximately 300 μm. The use of a 600 μm length and 300 μm width provides an opening within the known pore sizes for bone growth and provides a surface area of roughly 1.8 square millimeters, allowing high strength bone growth to form. Alternative embodiments may contain interconnections with a cross sectional area of 1.0 to 15.0 times the cross-sectional area of a pore with a diameter of 300 μm. Other embodiments may contain interconnections with a cross sectional area of 1.0 to 15.0 times the cross-sectional area of a pore with a diameter of 900 μm.

The MRDD unit cell also has the advantage of providing at least two sets of substantially homogenous pore or opening sizes in a lattice structure. In some embodiments, a first set of pores have a width of about 200 μm to 900 μm and a second set of pores have a width of about 1 to 15 times the width of the first set of pores. In some embodiments, a first set of pores can be configured to promote the growth of osteoblasts and a second set of pores can be configured to promote the growth of osteons. Pores sized to promote osteoblast growth can have a width of between and including about 100 μm to 900 μm. In some embodiments, pores sized to promote osteoblast growth can have a width that exceeds 900 μm. Pores sized to promote the growth of osteons can have a width of between and including about 100 μm to 13.5 mm. In some embodiments, pores sized to promote osteon growth can have a width that exceeds 13.5 mm.

In some embodiments, it is beneficial to include a number of substantially homogenous larger pores and a number of substantially homogenous smaller pores, where the number of larger pores is selected based on a ratio relative to the number of smaller pores. For example, some embodiments have one large pore for every one to twenty-five small pores in the lattice structure. Some embodiments preferably have one large pore for every eight to twelve smaller pores. In some embodiments, the number of larger and smaller pores can be selected based on a percentage of the total number of pores in a lattice structure. For example, some embodiments can include larger pores for 4% to 50% of the total number of pores and smaller pores for 50% to 96% of the total number of pores. More preferably, some embodiments can include larger pores for about 8% to 13% of the total number of pores and smaller pores for about 87% to 92% of the total number of pores. It is believed that a lattice constructed with sets of substantially homogenous pores of the disclosed two sizes provides a lattice structure that simultaneously promotes osteoblast and osteon growth.

The MRDD unit cell may also be defined by the size of the interconnections when viewed from a side, top or bottom of a unit cell. The MRDD unit cell has the same appearance when viewed from a side, top or bottom, making the measurement in a side view representative of the others. When viewed from the side, as in FIG. 4, an MRDD unit cell displays four distinct diamond shaped interconnections with substantially right angles. The area of each interconnection is smaller when viewed in the lateral direction than from a direction normal to the planar direction of each interconnection, but the area when viewed in the lateral direction can represent the area available for bone to grow in that direction. In some embodiments, it may be desirable to index the properties of the unit cell and lattice based on the area of the interconnections when viewed from the top, bottom or lateral directions.

In some embodiments of the lattice structures disclosed herein, the central void is larger than the length or width of the interconnections. Because the size of each interconnection can be substantially the same in a repeating MRDD structure, the resulting lattice can be comprised of openings of at least two discrete sizes. In some embodiments, it is preferable for the diameter of the central void to be approximately two times the length of the interconnections. In some embodiments, it is preferable for the diameter of the central void to be approximately four times the width of the interconnections.

In some embodiments, the ratio between the diameter of the central void and the length or width of the interconnections can be changed to create a structural lattice of a particular strength. In these embodiments, there is a correlation where the ratio between the central void diameter and the length or width of the interconnections increases as the strength of the structural lattice increases.

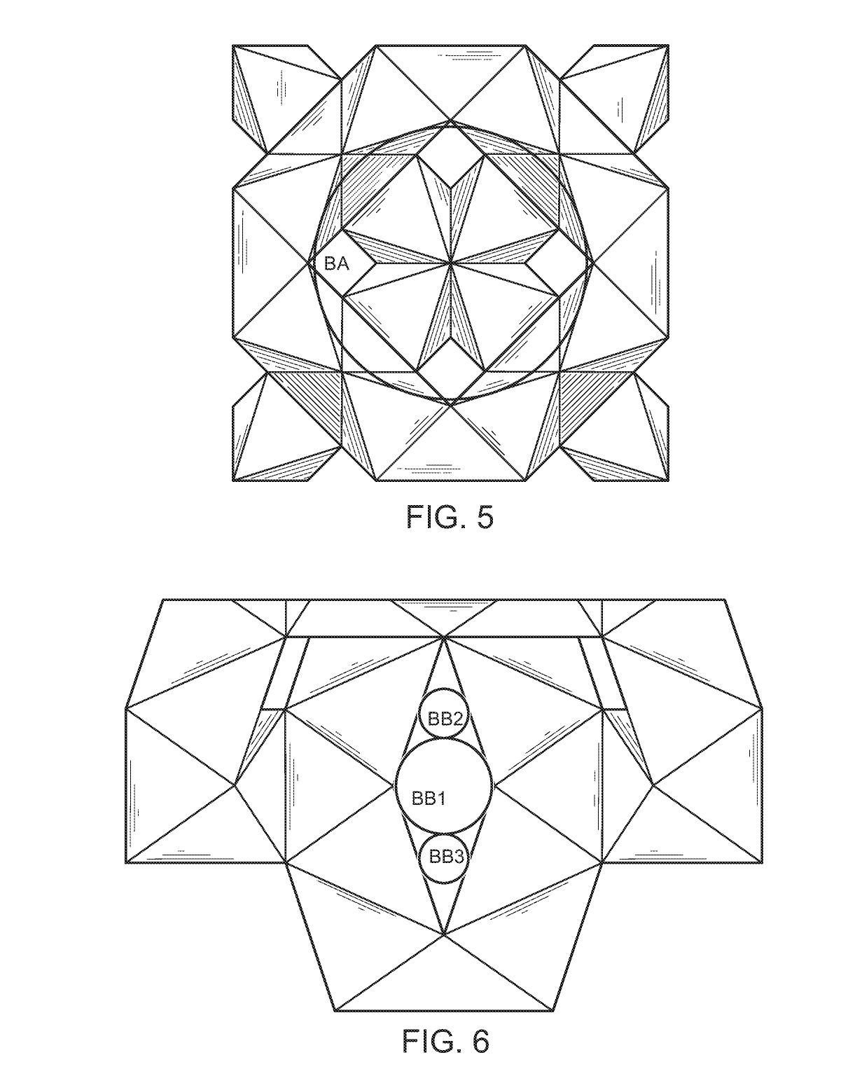

It is also believed that a lattice using a repeating radial dodeca-rhombus (hereinafter “RDDR”) unit cell can present a preferable combination of stiffness, strength, fatigue resistance, and conditions for bone ingrowth. In some embodiments, the repeating RDDR lattice comprises titanium or a titanium alloy. In FIG. 7 is an isometric view of a single RDDR unit cell 20 containing a full RDDR structure. In FIG. 8 is a side view of a single RDDR unit cell 20 showing the configuration of interconnections when viewed from a lateral direction. A top or bottom view of the RDDR unit cell 20 would be substantially the same as the side view depicted in FIG. 8.

As used herein, an RDDR unit cell 20 is a three-dimensional shape comprising a central node with radial struts and mirrored struts thereof forming twelve rhombus shaped structures. The node is preferably an octahedron, more specifically a square bipyramid (i.e., a pyramid and inverted pyramid joined on a horizontal plane). Each face of the node is preferably triangular and fixed to each face is a strut comprised of six triangular facets and two end faces. The central axis of each strut can be orthogonal or non-orthogonal relative to the planar surface of each node face. The central axis may follow the centroid of the strut. The RDDR is also characterized by a central node with one strut attached to each face, resulting in a square bipyramid node with eight struts attached.

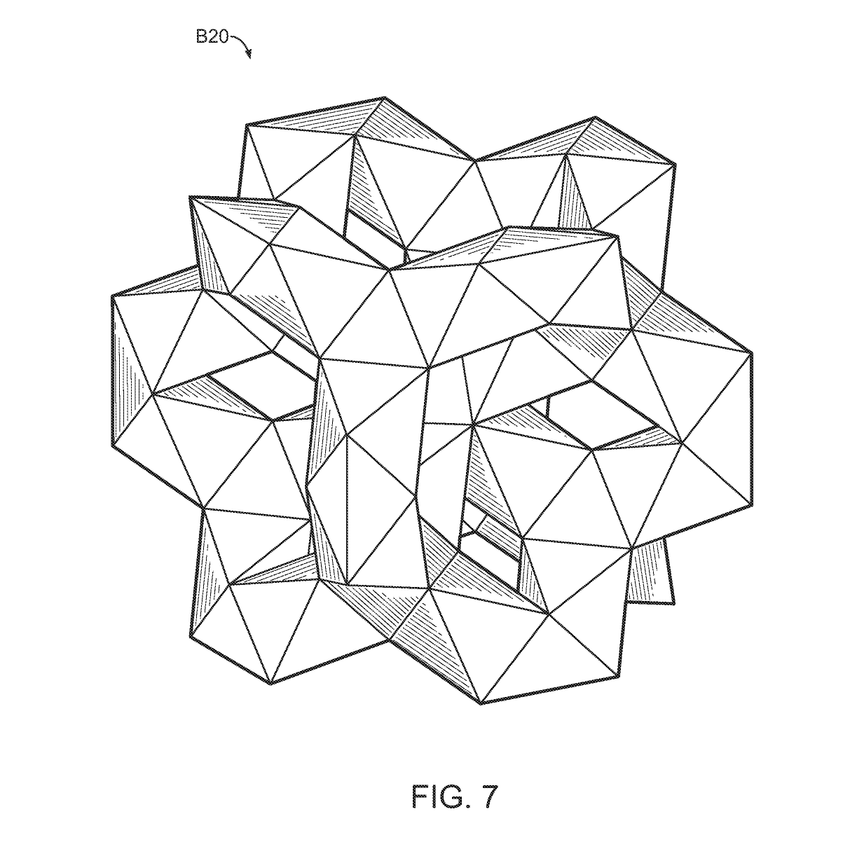

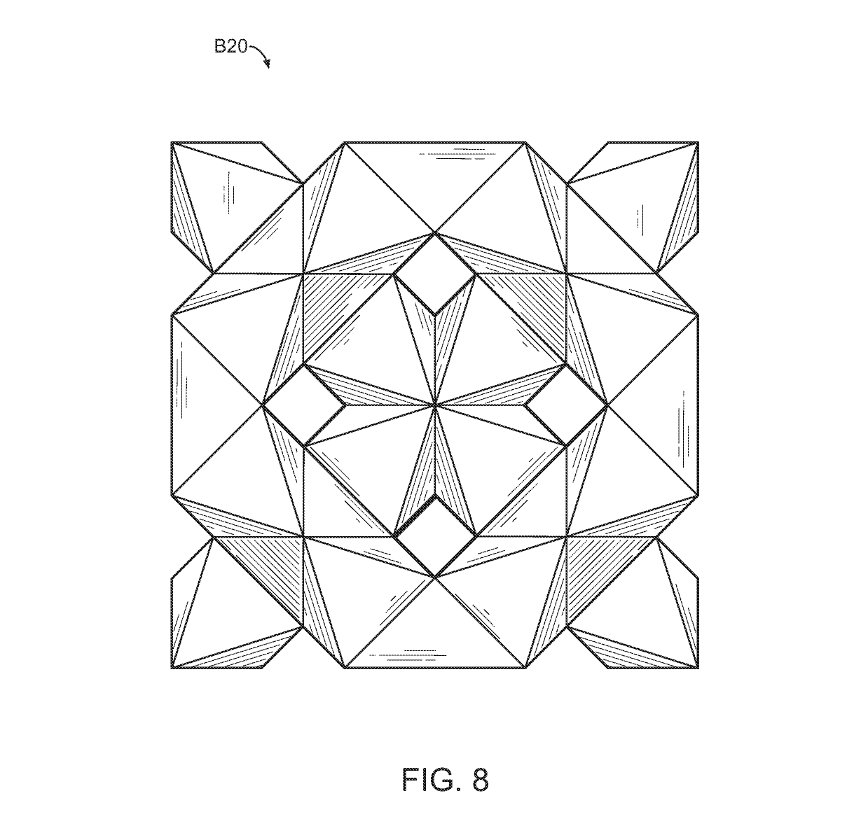

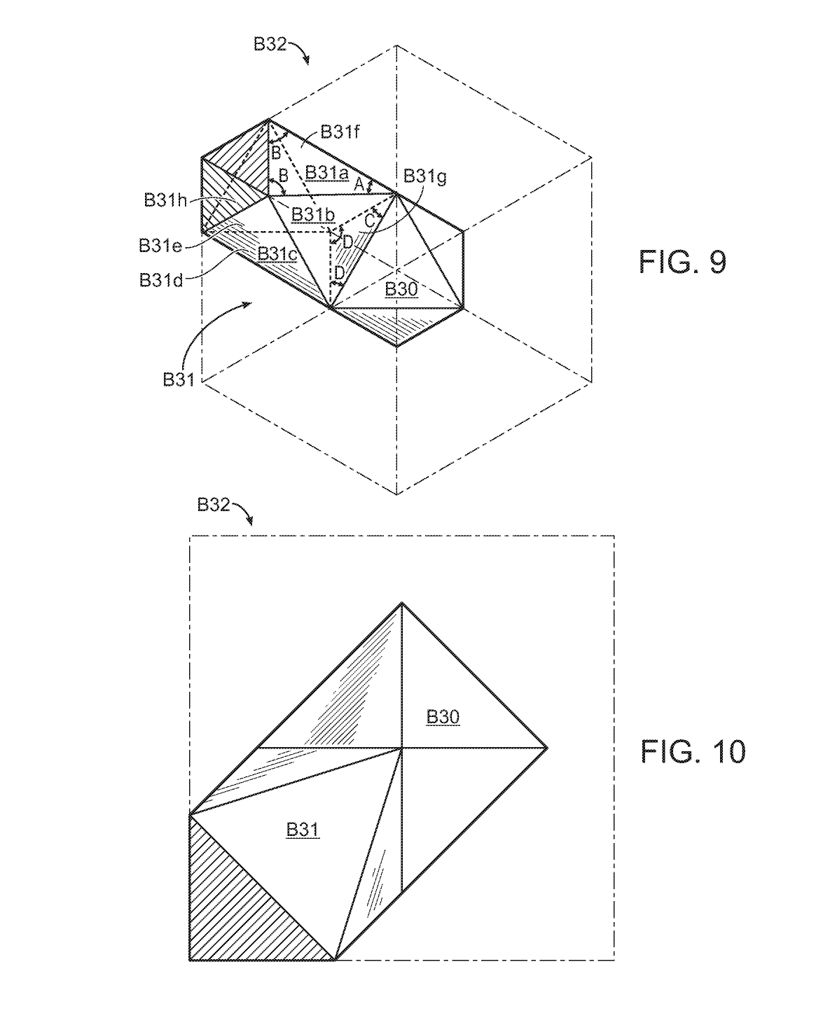

Examples of node and strut combinations are shown in FIGS. 9-13. FIG. 9 depicts an isometric view of a single node 30 with a single strut 31 attached. The node 30 is a square bipyramid oriented so that two peaks face the top and bottom of a volume 32 defining the bounds of the node 30 and any attached strut(s) 31. The node 30 is oriented so that the horizontal corners are positioned at their closest point to the lateral sides of the volume 32. The strut 31 extends from a node 30 face to the corner of the volume 32 defining the bounds of the node and attached struts. In FIG. 9, the central axis of the strut 31 is 45 degrees above the horizontal plane where the node's planar face is 45 degrees above a horizontal plane.

FIG. 9 also details an octahedron strut 31, where dashed lines show hidden edges of the strut. The strut 31 is an octahedron with an elongate portion of six substantially similar elongate faces and two end faces. The elongate faces 31a, 31b, 31c, 31d, 31e and 31f of the strut 31 define the outer surface of the strut's elongate and somewhat cylindrical surface. Each of the elongate faces 31a, 31b, 31c, 31d, 31e and 31f is an isosceles triangle with a first internal angle, angle A, and a second internal angle, angle B, where angle B is greater than angle A. The strut 31 also has two end faces 31f, 31g, which are isosceles triangles that are substantially similar to one another, having a first internal angle, angle C, and a second internal angle, angle D, and where angle D is greater than angle C. When comparing the internal angles of the elongate faces 31a, 31b, 31c, 31d, 31e and 31f to the end faces 31f and 31g, angle C is greater than angle A.

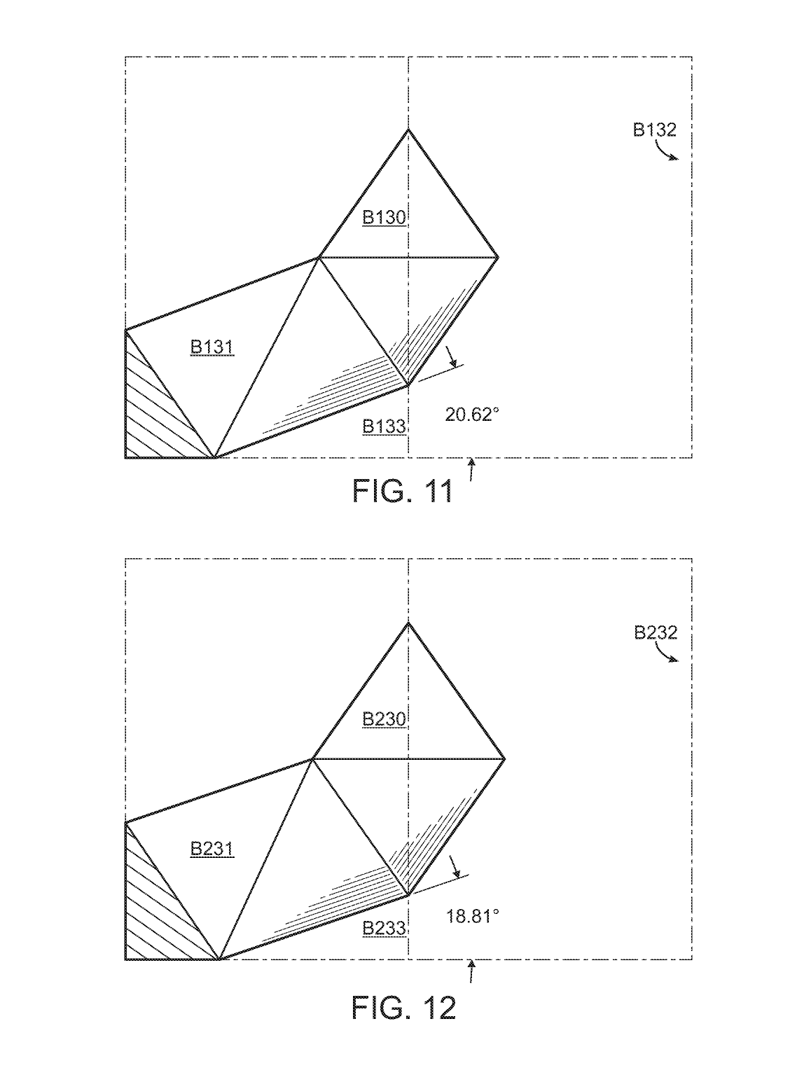

In FIG. 10 is a side view of the node 30 and strut 31 combination bounded by volume 32. In the side view, the height of the node 30 compared to the height of the cube 32 can be compared easily. FIGS. 11-13 depict side views of node and strut combinations, viewed from a corner of the volume rather than a wall or face, and where the combinations have been modified from FIGS. 9-10 to change the volumetric density of the resulting unit cell. In FIG. 11, the height of the node 130 has increased relative to the height of the volume 132. Since the distal end of the strut 131 is fixed by the location of a corner of the volume 132, the strut 131 must change its angle relative to its attached node face so that it becomes non-orthogonal. The node 130 and strut 131 combination, where the angle of the strut 131 from a horizontal plane is about 20.6 degrees, would be appropriate for a lattice structure with an elastic modulus of approximately 3 GPa, for a lattice structure comprising, for example, Ti4Al6V and a 2 mm×2 mm×2 mm unit cell.

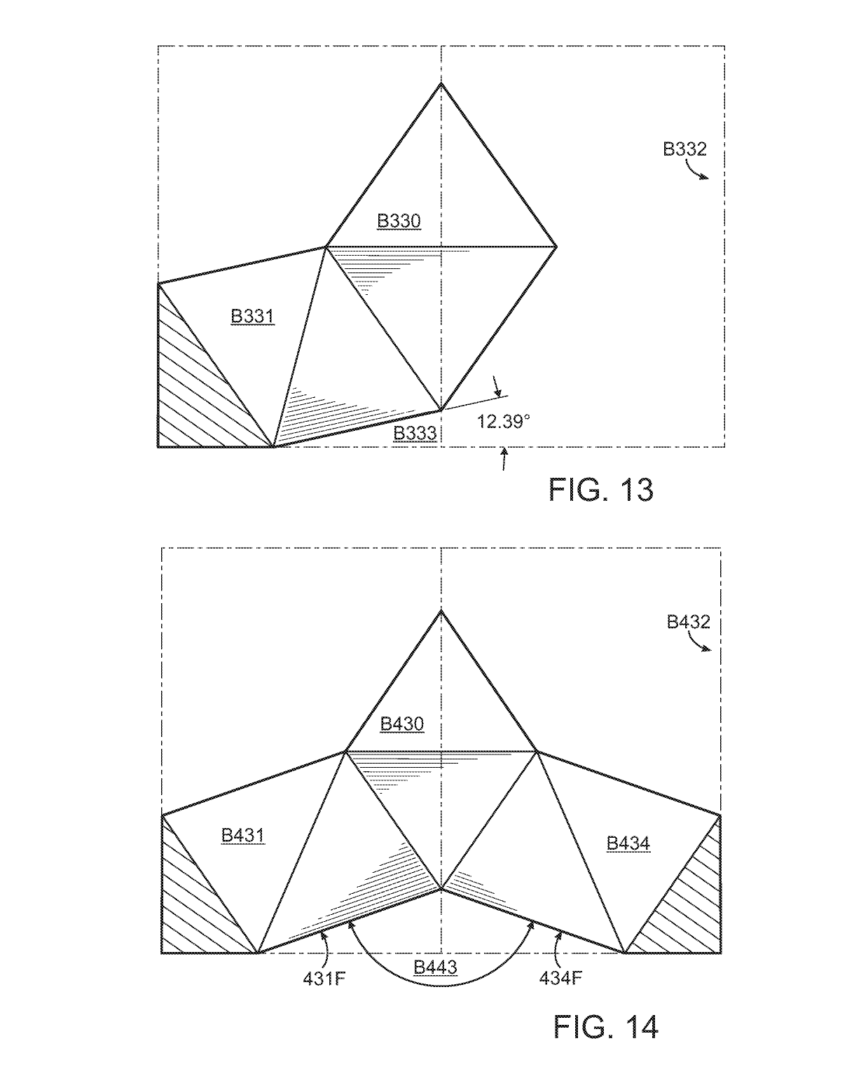

In FIG. 12, the height of the node 230 relative to the height of the cube 232 has been increased over the ratio of FIG. 11 to create a node 230 and strut 231 combination that would be appropriate for a lattice structure with an elastic modulus of approximately 4 GPa, for a lattice structure comprising, for example, Ti4Al6V and a 2 mm×2 mm×2 mm unit cell. As the height of the node 230 increases, the angle between the strut 231 and a horizontal plane decreases to about 18.8 degrees. As the height of the node 230 increases, the size of the node faces also increase so that the size of the strut 231 increases. While the distal end of the strut 231 is fixed to the corner of the volume 232, the size of the distal end increases to match the increased size of the node face to maintain a substantially even strut diameter along its length. As the node and strut increase in volume, the volumetric density increases, as does the elastic modulus. In FIG. 13, the height of the node 330 relative to the height of the volume 332 has been increased over the ratio of FIG. 13 to create a node 330 and strut 331 combination that would be appropriate for a lattice structure with an elastic modulus of approximately 10 GPa, for a lattice structure comprising, for example, Ti4Al6V and a 2 mm×2 mm×2 mm unit cell. In this configuration, the angle 333 between the strut 331 and a horizontal plane decreases to about 12.4 degrees and the volumetric density increases over the previous examples. The single node and strut examples can be copied and/or mirrored to create unit cells of appropriate sizes and characteristics. For instance, the angle between the strut and a horizontal plane could be increased to 25.8 degrees to render a lattice with a 12.3% volumetric density and an elastic modulus of about 300 MPa. While a single node and single strut were shown in the examples for clarity, multiple struts may be attached to each node to create an appropriate unit cell or sub-unit cell.

Adjacent struts extending from adjacent node faces on either the upper half or lower half of the node have an angle from the horizontal plane and a lateral separation angle defined by an angle between the strut directions of adjacent struts. In the MRDD and RDDR structures, adjacent struts have an external edge or face of the elongate portion extending closest to the relevant adjacent strut. The lateral separation angle, as used herein, generally refers to the angle between an external edge or face of the elongate portion of a strut extending closest to the relevant adjacent strut. In some embodiments, a lateral separation angle defined by a line extending between the center of the strut end faces or a line defined by the center of mass of the struts can be used in reference to a similar calculation for an adjacent strut.

The lateral separation angle is the angle between the nearest face or edge of a strut to an adjacent strut. The lateral separation angle can be measured as the smallest angle between the nearest edge of a strut to the nearest edge of an adjacent strut, in a plane containing both strut edges. The lateral separation angle can also be measured as the angle between the nearest face of a strut to the nearest face of an adjacent strut in a plane normal to the two strut faces. In embodiments without defined strut edges or strut faces, the lateral separation angle can be measured as an angle between the nearest portion of one strut to the nearest portion of an adjacent strut. For a unit cell in a cubic volume, as the strut angle from the horizontal plane decreases, the lateral separation angle approaches 90 degrees. For a unit cell in a cubic volume, as the strut angle from the horizontal plane increases, the lateral separation angle approaches 180 degrees. In some embodiments, it is preferable to have a lateral separation angle greater than 109.5 degrees. In some embodiments, it is preferable to have a lateral separation angle of less than 109.5 degrees. In some embodiments, it is preferable to have a lateral separation angle of between and including about 108 degrees to about 156 degrees. In some embodiments, it is more preferable to have a lateral separation angle of between and including 111 degrees to 156 degrees. In some embodiments, it is more preferable to have a lateral separation angle of between and including 108 degrees to 120 degrees. In some embodiments, it is most preferable to have a lateral separation angle of between and including about 111 degrees to 120 degrees. In some embodiments, it is more preferable to have a lateral separation angle of between and including 128 degrees to 156 degrees. FIG. 14 depicts a side view, viewed from a corner of the cube 432, of a single node 430 with two adjacent struts 431, 434 attached, and where the lateral separation angle 443 is identified. When measured from the nearest edge of a strut to the nearest edge of an adjacent strut, the lateral separation angle 443 is about 116 degrees.

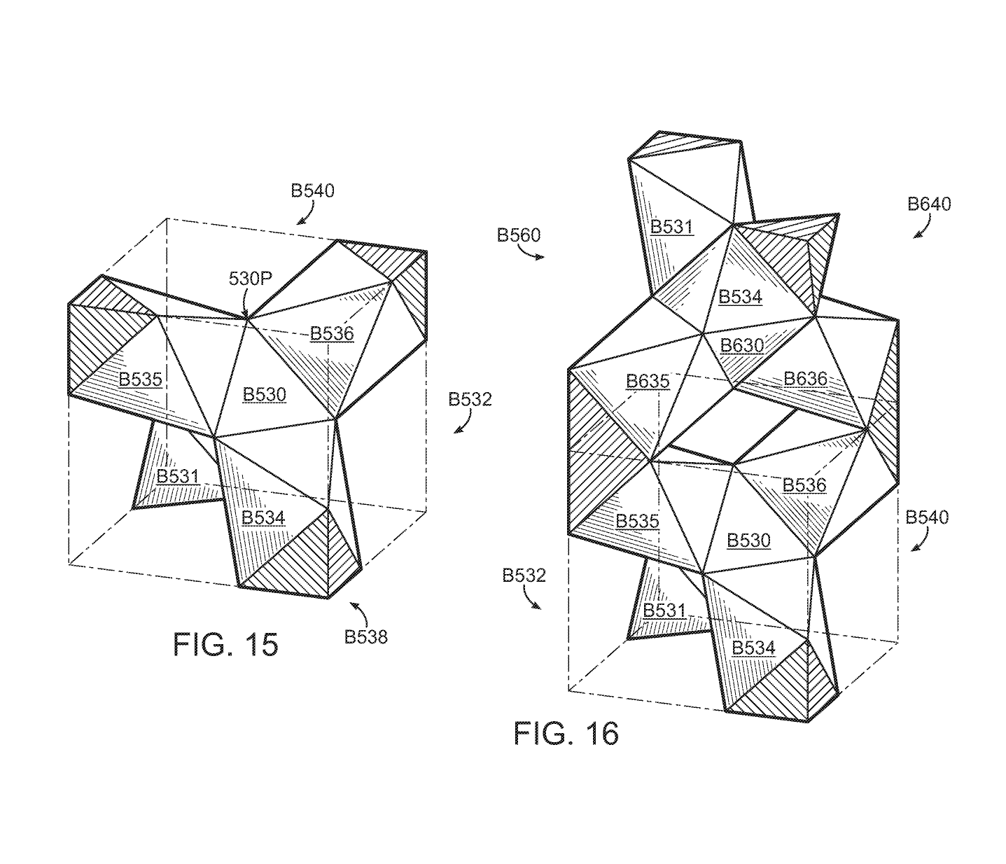

In some embodiments, a unit cell is built up from multiple sub-unit cells fixed together. FIG. 15 depicts an isometric view of an exemplary sub-unit cell comprising a single node and four struts. FIG. 16 depicts an isometric view of two sub-unit cells in a stacked formation where the upper sub-unit cell is inverted and fixed to the top of the lower sub-unit cell. FIG. 17 depicts an isometric view of eight sub-unit cells stacked together to form a single RDDR unit cell.

In FIG. 15, the node 530 is a square bipyramid, oriented so that the two peaks face the top and bottom of a cubic volume 532. In some embodiments, the volume 532 can be a cuboid volume, a hexahedron volume, an amorphous volume or of a volume with one or more non-orthogonal sides. The peaks refer to the point where four upper faces meet and the point where four lower faces meet. The node 530 is oriented so that the horizontal vertices face the lateral sides of the cubic volume 532. The strut 531 is fixed to a lower face of the node 530 face on its proximate end, and it extends to the nearest corner of the cubic volume 532 at its distal end. The distal end of the strut 531 can remain fixed to the cubic volume 532 even if the node 530 changes in size to adjust the sub-unit cell properties.

On the lower face of the node 530 that is opposite the face which strut 531 is fixed, the proximate end of strut 534 is fixed to the node 530. The strut 534 extends to the nearest corner of cubic volume 532 at its distal end. The strut 535 is fixed on its proximate end to an upper node 530 face directed about 90 degrees laterally from the node 530 face fixed to strut 531. The strut 535 extends to the nearest corner of the cubic volume 532 at its distal end. On the upper face of the node 530 that is opposite the face which strut 535 is fixed, the proximate end of strut 536 is fixed to the node 530. The strut 536 extends to the nearest corner of the cubic volume 532 at its distal end.

In some embodiments, the struts 531, 534-536 are octahedrons with triangular faces. The strut face fixed to a node 530 face can be substantially the same size and orientation of the node 530 face. The strut face fixed to the nearest corner of the cube 532 can be substantially the same size as the strut face fixed to the node 530 and oriented on a substantially parallel plane. The remaining six faces can be six substantially similar isosceles triangles with a first internal angle and a second internal angle larger than said first internal angle. The six substantially similar isosceles triangles can be fixed along their long edges to an adjacent and inverted substantially similar isosceles triangle to form a generally cylindrical shape with triangular ends.

When forming a sub-unit cell 540, it can be beneficial to add a one-eighth node 538 to each corner of the cube 532 fixed to a strut 531, 534-536. When replicating the sub-unit cell 540, the one-eighth node 538 attached to each strut end is combined with one-eighth nodes from adjacent sub-unit cells to form nodes located between the struts of adjacent sub-unit cells.

In FIG. 16 is a first sub-unit cell 540 fixed to a second sub-unit cell 640 to form a quarter unit cell 560 used in some embodiments. The second sub-unit cell 640 comprises a square bipyramid node 630, oriented so that the two peaks face the top and bottom of a cubic volume. The node 630 is oriented so that the horizontal vertices face the lateral sides of the cubic volume. The strut 635 is fixed to a lower face of the node 630 face on its proximate end and extends to the nearest corner of the cubic volume at its distal end.

On the lower face of the node 630 opposite the face which strut 635 is fixed, the proximate end of strut 636 is fixed to the node 630. The strut 636 extends to the nearest corner of cubic volume at its distal end. The strut 634 is fixed on its proximate end to an upper node 630 face directed about 90 degrees laterally from the node 630 face fixed to strut 635. The strut 634 extends to the nearest corner of the cubic volume at its distal end. On the upper face of the node 630 opposite the face which strut 634 is fixed, the proximate end of strut 631 is fixed to the node 630. The strut 631 extends to the nearest corner of the cubic volume at its distal end.

The first sub-unit 540 is used as the datum point in the embodiment of FIG. 16, however, it is appreciated that the second sub-unit cell 640 or another point could also be used as the datum point. Once the first sub-unit cell 540 is fixed in position, it is replicated so that the second sub-unit cell 640 is substantially similar to the first. The second sub-unit cell 640 is rotated about its central axis prior to being fixed on the top of the first unit-cell 540. In FIG. 16, the second sub-unit cell 640 is inverted to achieve the proper rotation, however, other rotations about the central axis can achieve the same result. The first sub-unit cell 540 fixed to the second sub-unit cell 640 forms a quarter unit cell 560 that can be replicated and attached laterally to other quarter unit cells to form a full unit cell.

Alternatively, a full unit cell can be built up by fixing a first group of four substantially similar sub-unit cells together laterally to form a square, rectangle or quadrilateral when viewed from above. A second group of four substantially similar sub-unit cells rotated about their central axis can be fixed together laterally to also form a square, rectangle or quadrilateral when viewed from above. The second group of sub-unit cells can be rotated about their central axis prior to being fixed together laterally or inverted after being fixed together to achieve the same result. The second group is then fixed to the top of the first group to form a full unit cell.

In FIG. 17 is an example of a full unit cell 770 formed by replicating the sub-unit cell 540 of FIG. 15. The cube 532 defining the bounds of the sub-unit cell 540 is identified as well as the node 530 and struts 531, 534-536 for clarity. The full unit cell 770 of FIG. 17 can be formed using the methods described above or using variations within the inventive concept.

Each strut extending from the node, for a given unit cell, can be substantially the same length and angle from the horizontal plane, extending radially from the node. At the end of each strut, the strut is mirrored so that struts extending from adjacent node faces form a rhombus shaped opening. Because the struts can be non-orthogonal to the node faces, rhombuses of two shapes emerge. In this configuration, a first group of four rhombuses extend radially from the node oriented in vertical planes. The acute angles of the first group of rhombuses equal twice the strut angle from the horizontal plane and the obtuse angles equal 180 less the acute angles. Also in this configuration is a second group of eight rhombuses extending radially so that a portion of the second group of eight rhombuses fall within the lateral separation angle between adjacent struts defining the first group of four rhombuses. The acute angles of the second group of rhombuses can be about the same as the lateral separation angle between adjacent struts that define the first group of four rhombuses and the obtuse angles equal 180 less the acute angles.

The characteristics of a scaffold may also be described by its surface area per volume. For a 1.0 mm×1.0 mm×1.0 mm solid cube, its surface area is 6.0 square mm. When a 1.0 cubic mm structure is comprised of a lattice structure rather than a 100% volumetric density material, the surface area per volume can increase significantly. In low volumetric density scaffolds, the surface area per volume increases as the volumetric density increases. In some embodiments, a scaffold with a volumetric density of 30.1% would have a surface area of 27.4 square mm per cubic mm. In some embodiments, if the volumetric density was decreased to 27.0%, the lattice would have a surface area of 26.0 square mm per cubic mm and if the volumetric density were decreased to 24.0%, the lattice would have a surface area of 24.6 square mm per cubic mm.

The MRDD and RDDR structures disclosed herein also have the advantage of an especially high modulus of elasticity for a given volumetric density. When used as a lattice or scaffold, an implant with an adequate modulus of elasticity and a low volumetric density can be achieved. A low volumetric density increases the volume of the implant available for bone ingrowth.

In Table 1, below, are a number of example lattice configurations of various lattice design elastic moduli. The lattice configurations shown in Table 1 can comprise, for example, Ti4Al6V and a 2 mm×2 mm×2 mm unit cell. An approximate actual elastic modulus was given for each example, representing a calculated elastic modulus for that lattice after going through the manufacturing process. The lattice structures and implants disclosed herein can be designed to a design elastic modulus in some embodiments and to an approximate actual elastic modulus in other embodiments. One advantage of the presently disclosed lattice structures is that the approximate actual elastic modulus is much closer to the design elastic modulus than has been previously achieved. During testing, one embodiment of a lattice was designed for a 4.0 GPa design elastic modulus. Under testing, the lattice had an actual elastic modulus of 3.1 GPa, achieving an actual elastic modulus within 77% of the design elastic modulus.

For each lattice design elastic modulus, a volumetric density, ratio of design elastic modulus to volumetric density, surface area in mm², ratio of surface area to volumetric density and ratio of surface area to lattice design elastic modulus is given.

TABLE 1
Table of example lattice structures based on lattice design elastic modulus in GPa

| | | | Ratio of | | | |
|---|---|---|---|---|---|---|
| | | | Design Elastic | | Ratio of | Ratio of |
| Lattice Design | Approx. Actual | Volumetric | Modulus to | Surface | Surface Area | Surface Area to |
| Elastic Modulus | Elastic Modulus | Density | Volumetric | Area | to Volumetric | Lattice Design |
| (GPa) | (GPa) | (percent) | Density | (mm²) | Density | Elastic Modulus |
| 0.3 | 0.233 | 18.5 | 1.6 | 22.5 | 121.5 | 74.9 |
| 3 | 2.33 | 29.9 | 10.0 | 27.5 | 92.2 | 9.2 |
| 4 | 3.10 | 33.4 | 12.0 | 28.8 | 86.4 | 7.2 |
| 5 | 3.88 | 36.4 | 13.8 | 29.9 | 82.2 | 6.0 |
| 6 | 4.65 | 38.8 | 15.5 | 30.7 | 79.1 | 5.1 |
| 7 | 5.43 | 40.8 | 17.2 | 31.3 | 76.9 | 4.5 |
| 8 | 6.20 | 42.1 | 19.0 | 31.8 | 75.4 | 4.0 |
| 9 | 6.98 | 43.2 | 20.8 | 32.1 | 74.3 | 4.0 |

In some of the embodiments disclosed herein, the required strut thickness can be calculated from the desired modulus of elasticity. Using the following equation, the strut thickness required to achieve a particular elastic modulus can be calculated for some MRDD and RDDR structures:

Strut Thickness=(−0.0035*(Ê2))+(0.0696* E)+0.4603

In the above equation, “E” is the modulus of elasticity. The modulus of elasticity can be selected to determine the required strut thickness required to achieve that value or it can be calculated using a preselected strut thickness. The strut thickness is expressed in mm and represents the diameter of the strut. The strut thickness may be calculated using a preselected modulus of elasticity or selected to determine the modulus of elasticity for a preselected strut thickness.

In some embodiments, the unit cell can be elongated in one or more directions to provide a lattice with anisotropic properties. When a unit cell is elongated, it generally reduces the elastic modulus in a direction normal to the direction of the elongation. The elastic modulus in the direction of the elongation is increased. It is desirable to elongate cells in the direction normal to the direction of new bone growth contained within the interconnections, openings and central voids (if any). By elongating the cells in a direction normal to the desired direction of reduced elastic modulus, the shear strength in the direction of the elongation may be increased, providing a desirable set of qualities when designing a structural scaffold. Covarying the overall stiffness of the scaffold may augment or diminish this effect, allowing variation in one or more directions.

In some embodiments, the sub-unit cells may be designing by controlling the height of the node relative to the height of the volume that defines the sub-unit cell. Controlling the height of the node can impact the final characteristics and appearance of the lattice structure. In general, increasing the height of the node increases the strut thickness, increases the volumetric density, increases the strength and increases the elastic modulus of the resulting lattice. When increasing the height of the node, the width of the node can be held constant in some embodiments or varied in other embodiments.

In some embodiments, the sub-unit cells may be designing by controlling the volume of the node relative to the volume that defines the sub-unit cell. Controlling the volume of the node can impact the final characteristics and appearance of the lattice structure. In general, increasing the volume of the node increases the strut thickness, increases the volumetric density, increases the strength and increases the elastic modulus of the resulting lattice. When increasing the volume of the node, the width or height of the node could be held constant in some embodiments.

In Table 2, below, are a number of example lattice configurations of various lattice design elastic moduli. The lattice configurations shown in Table 2 can comprise, for example, Ti4Al6V and a 2 mm×2 mm×2 mm unit cell. An approximate actual elastic modulus was given for each example, representing a calculated elastic modulus for that lattice after going through the manufacturing process. The lattice structures and implants disclosed herein can be designed to a design elastic modulus in some embodiments and to an approximate actual elastic modulus in some embodiments. For each lattice design elastic modulus, a lattice approximate elastic modulus, a node height, a volumetric density, a node volume, a ratio of node height to volumetric density, a ratio of node height to lattice design elastic modulus and a ratio of volumetric density to node volume is given.

TABLE 2
Table of example lattice structures based on lattice design elastic modulus in GPa

| | Lattice | | | | Ratio of | Ratio of | Ratio |
|---|---|---|---|---|---|---|---|
| Lattice Design | Approx. Actual | Node | Volumetric | Node | Node Height | Node Height to | of Vol. |
| Elastic Modulus | Elastic Modulus | Height | Density | Volume | to Vol. | Lattice Design | Density to |
| (GPa) | (GPa) | (mm) | (percent) | (mm3) | Density | Elastic Modulus | Node Volume |
| 0.30 | 0.23 | 0.481 | 18.5 | 0.0185 | 2.60 | 1.60 | 9.98 |
| 3.00 | 2.33 | 0.638 | 29.9 | 0.0432 | 2.14 | 0.21 | 6.91 |
| 4.00 | 3.10 | 0.683 | 33.4 | 0.0530 | 2.05 | 0.17 | 6.29 |
| 5.00 | 3.88 | 0.721 | 36.4 | 0.0624 | 1.98 | 0.14 | 5.82 |
| 6.00 | 4.65 | 0.752 | 38.8 | 0.0709 | 1.94 | 0.13 | 5.48 |
| 7.00 | 5.43 | 0.776 | 40.8 | 0.0779 | 1.90 | 0.11 | 5.23 |
| 8.00 | 6.20 | 0.793 | 42.1 | 0.0831 | 1.88 | 0.10 | 5.07 |
| 9.00 | 6.98 | 0.807 | 43.2 | 0.0877 | 1.87 | 0.09 | 4.93 |

Some embodiments of the disclosed lattice structures are particularly useful when provided within an elastic modulus range between an including 0.375 GPa to 4 GPa. Some embodiments, more preferably, include a lattice structure with an elastic modulus between and including 2.5 GPa to 4 GPa. Some embodiments include a lattice structure with a volumetric density between and including 5% to 40%. Some embodiments, more preferably, include a lattice structure with a volumetric density between and including 30% to 38%.

The lattice structures disclosed herein have particularly robust loading and fatigue characteristics for low volumetric density ranges and low elastic moduli ranges. Some embodiments of the lattice structures have a shear yield load and a compressive yield load between and including 300 to 15000N in static and dynamic loading up to 5,000,000 cycles at 5 Hz. Some embodiments have a compressive shear strength and an axial load between and including 300 to 15000N in static and dynamic loading up to 5,000,000 cycles at 5 Hz. Some embodiments have a shear strength and an axial load between and including 300 to 15000N in static and dynamic loading up to 5,000,000 cycles at 5 Hz. Some embodiments have a torsional yield load up to 15 Nm.

In one example, the inventive lattice structure has a volumetric density of between and including 32% to 38%, an elastic modulus between and including 2.5 GPa to 4 GPa and a shear strength and an axial load between and including 300 to 15000N in static and dynamic loading up to 5,000,000 cycles at 5 Hz. Some examples include a first set of substantially homogeneous openings with a width of about 200 μm to 900 μm and a second set of substantially homogenous openings with a width of about 1 to 15 times the width of the first set of openings, where the number of openings in the second set are provided at a ratio of about 1:8 to 1:12 relative to the number of openings in the first set.

The disclosed structures can also have benefits when used in applications where osteointegration is not sought or undesirable. By including a growth inhibiting coating or skin on a structure, the lattice disclosed herein can be used to provide structural support without providing a scaffold for bone growth. This may be desirable when used in temporary implants or medical devices that are intended to be removed after a period of time.

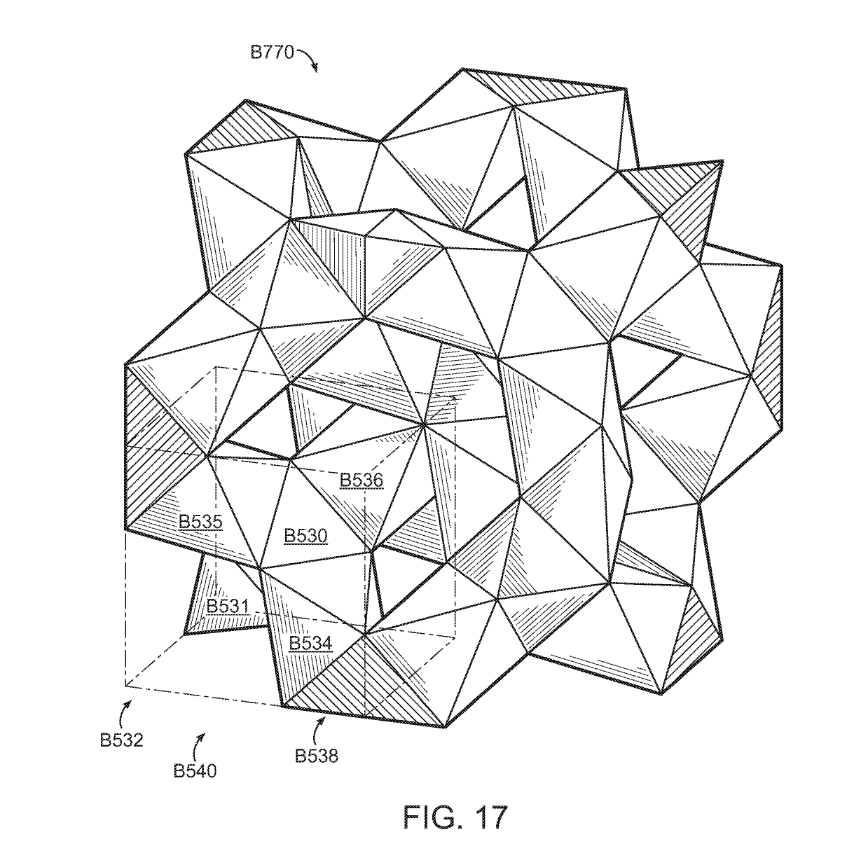

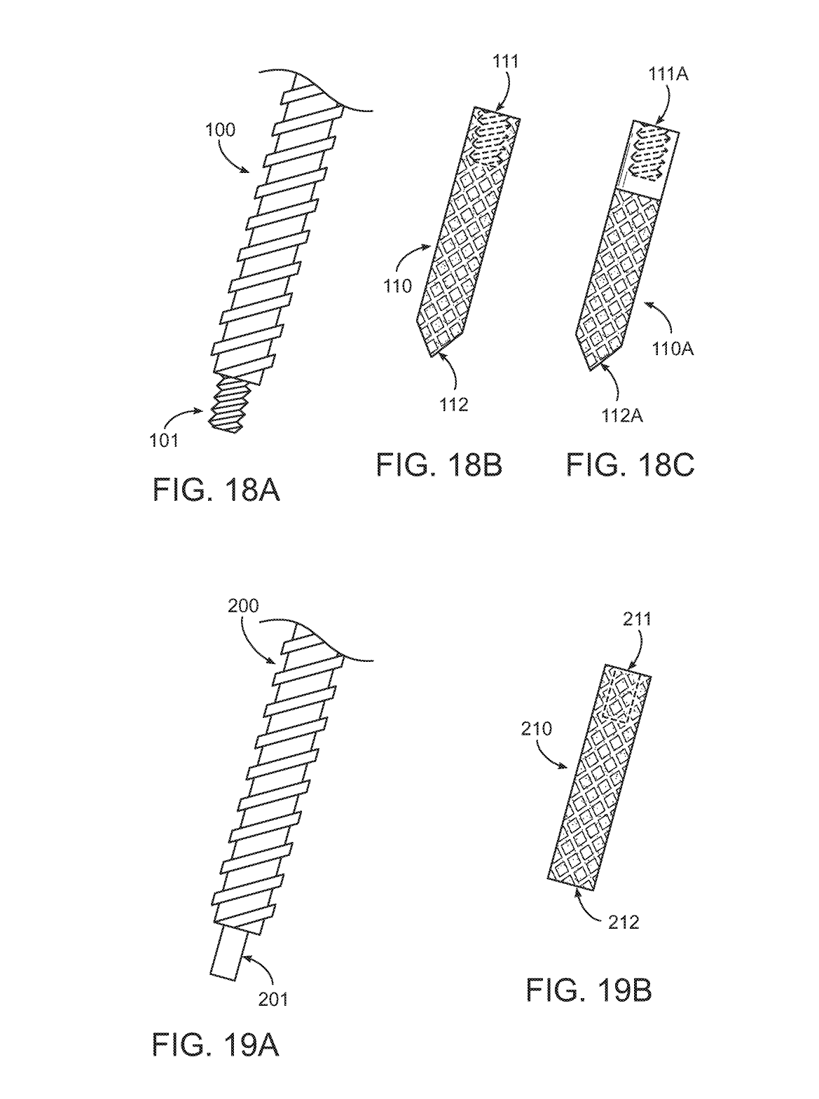

In FIG. 18A is a first exemplary embodiment of an upper bone anchor 100 configured to receive a detachable lower bone anchor. The upper bone anchor 100 can be generally elongate in shape and configured with an attachment point 101 at its lower end.

The terms “upper” and “lower” are exemplary and used to describe the example orientations disclosed herein. The exemplary embodiments could be used in any orientation, making the terms upper and lower only relevant to the example orientations. The term “bone anchor,” as used herein, refers to any device or medical implant that can be fixed relative to bone or tissue during or after implantation in a patient. A patient refers to any living organism, such as a human being or an animal. As used herein, the term “bone anchor” can refer to a number of devices and medical implants, including but not limited to, bone screws, staples, lattice segments, suture anchors, wings, barbs, and tethers.

The upper bone anchor 100 can be threaded on an circumferential surface to provide initial fixation to bone. As used herein, “threaded” refers to a generally helix shaped thread on a shaft configured to provide a force in the axial direction when rotated. In some embodiments, the upper bone anchor 100 can be non-threaded on part or all of its circumferential surface or threaded on all or part of its circumferential surface. In some embodiments, at least a portion of the upper bone anchor 100 is comprised of a lattice structure. In some embodiments, the entire upper bone anchor 100 is comprised of a lattice material.

The attachment point 101 located towards the bottom of the upper bone anchor 100 can be threaded along its circumferential surface to facilitate the attachment of a correspondingly threaded component. While the attachment point 101 shown is a male threaded fastener, it is appreciated that the attachment point 101 could be substituted with other fasteners, including but not limited to, a female threaded fastener, a pin, a press fit, a clip, a snap, or other structures only achievable by 3D printing and/or another additive manufacturing process. The attachment point 101 is preferably configured to accept a corresponding component that can be detached during revision surgery. However, in some embodiments, the attachment point 101 can be configured to accept a corresponding component that is permanently fixed to the upper bone anchor 100. Alternatively, the upper bone anchor 100 may be configured to detach at any point to achieve a similar detachable function.

The exemplary embodiments of bone anchors disclosed herein do not depict a head, which could be located on the distal end of the upper bone anchor 100 from the attachment point 101. There are various types of heads or screw heads known in the art that could transmit the torque from an insertion tool to the upper bone anchor 100 for implantation or removal. Such heads may be provided in multiple piece assemblies and may interface with other implants and surgical hardware.

In FIG. 18B is a first exemplary detachable lower bone anchor 110 configured to removably connect to the attachment point 101 on upper bone anchor 100. The attachment point 111 can be a threaded cylindrical opening in some embodiments. In some embodiments, a threaded area on the attachment point 111 can comprise a localized higher volumetric density area of only, for example, 0.5, 1 or 1.5 mm thick. A threaded area on the attachment area 101 or 111, in some embodiments, may be constructed using a thin wall of higher volumetric density material. A higher volumetric density area can include any material with a volumetric density of at least 85%. The lower bone anchor 110 is preferentially comprised, in part or completely, of a repeating unit cell lattice structure. The lower tip 112 of the lower bone anchor 110 may be comprised of a solid material or a higher volumetric density material in some embodiments to facilitate tissue distraction during implantation.

The lower bone anchor 110 can be configured to attach to a specific type of attachment point 101. In some embodiments, the lower bone anchor 110 can be threaded on its circumferential surface. If the lower bone anchor 110 is threaded, it is preferable for the thread pitch to be substantially similar to the thread pitch on the upper bone anchor (if also threaded).

In FIG. 18C is an alternative first exemplary detachable lower bone anchor 110A configured to removably connect to attachment point 101 on upper bone anchor 100. The lower bone anchor 110A is preferentially comprised, in part or completely, of a repeating unit cell lattice structure. The lower tip 112A of the lower bone anchor 110A may be comprised of a solid material in some embodiments to facilitate tissue distraction during implantation. Solid or substantially solid, as used herein when referring to an area of a bone screw, describes a material with a volumetric density greater than 85%. The solid or substantially solid material, when used on the lower tip 112A, does not need to extend significantly below the surface of the lower tip 112A and can be merely a solid or substantially solid shell or coating. In some embodiments, solid or substantially solid can refer to an area of the bone anchor that is configured to facilitate tissue distraction during implantation rather than a specific volumetric density. In some embodiments, solid or substantially solid can refer to a substantially smooth or relatively smooth, compared to the roughness of other areas of the bone screw or bone anchor, outer layer configured to facilitate tissue distraction during implantation

The lower bone anchor 110A can be configured to attach to a type of attachment point 101. For example, if the attachment point 101 is a threaded cylindrical protrusion, the lower bone anchor 110A could have a corresponding threaded cylindrical opening 111A for a threaded fit. In some embodiments, the lower bone anchor 110A can be threaded on its circumferential surface. If the lower bone anchor 110A is threaded, the thread pitch can be substantially similar to the thread pitch on the upper bone anchor (if also threaded), can be different or can progressively change in thread pitch.

In FIG. 19A is a second exemplary embodiment of an upper bone anchor 200 configured to receive a detachable lower bone anchor. The upper bone anchor 200 can be generally elongate in shape and configured with an attachment point 201 at its lower end. The upper bone anchor 200 can be threaded on its circumferential surface to provide initial fixation to bone. In some embodiments, the upper bone anchor 200 can be non-threaded on part or all of its circumferential surface or threaded on all or part of its circumferential surface. In some embodiments, at least a portion of the upper bone anchor 200 is comprised of a lattice structure. In some embodiments, the entire upper bone anchor 200 is comprised of a lattice material.

The attachment point 201 located towards the bottom of the upper bone anchor 200 can be configured to accept a corresponding fastener. While the attachment point 201 shown is a male fastener, it is appreciated that the attachment point 201 could be substituted with other fasteners, including but not limited to, a female fastener, a pin, a press, a clip or a snap. The attachment point 201 is preferably configured to accept a corresponding component that can be detached during revision surgery. However, in some embodiments, the attachment point 201 can be configured to accept a corresponding component that is permanently fixed to the upper bone anchor 200. Alternatively, the upper bone anchor 200 may be configured to detach at any point to achieve a similar detachable function.

In FIG. 19B is a second exemplary detachable lower bone anchor 210 configured to removably connect to the attachment point 201 on upper bone anchor 200. The lower bone anchor 210 is preferentially comprised, in part or completely, of a repeating unit cell lattice structure. The lower tip 212 of the lower bone anchor 210 may be comprised of a solid material in some embodiments to facilitate tissue distraction during implantation.

The lower bone anchor 210 can be configured to attach to a specific type of attachment point 201. For example, if the attachment point 201 is a cylindrical protrusion, the lower bone anchor 210 could have a corresponding cylindrical opening 211 to provide an interference fit. In some embodiments, the lower bone anchor 210 can be threaded on its circumferential surface. If the lower bone anchor 210 is threaded, it is preferable for the thread pitch to be substantially similar to the thread pitch on the upper bone anchor (if also threaded).

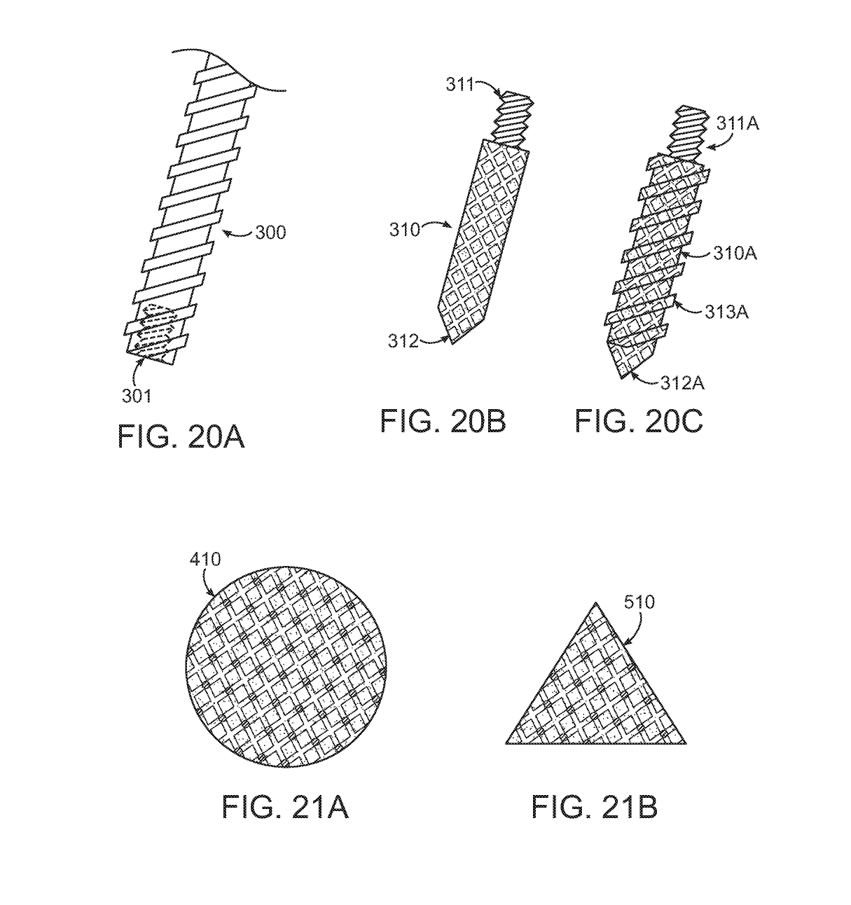

In FIG. 20A is a third exemplary embodiment of an upper bone anchor 300 configured to receive a detachable lower bone anchor. The upper bone anchor 300 can be generally elongate in shape and configured with an attachment point 301 at its lower end.

The upper bone anchor 300 can be threaded on an circumferential surface to provide initial fixation to bone. As used herein, “threaded” refers to a generally helix shaped thread on a shaft configured to provide a force in the axial direction when rotated. In some embodiments, the upper bone anchor 300 can be non-threaded on part or all of its circumferential surface or threaded on all or part of its circumferential surface. In some embodiments, at least a portion of the upper bone anchor 300 is comprised of a lattice structure. In some embodiments, the entire upper bone anchor 300 is comprised of a lattice material.

The attachment point 301 located towards the bottom of the upper bone anchor 300 can comprise a cylindrical opening threaded along its circumferential surface to facilitate the attachment of a correspondingly threaded component. While the attachment point 301 shown is a female threaded fastener, it is appreciated that the attachment point 301 could be substituted with other fasteners, including but not limited to, a male threaded fastener, a pin, a press fit, a clip, a snap, or other structures only achievable by 3D printing and/or another additive manufacturing process. The attachment point 301 is preferably configured to accept a corresponding component that can be detached during revision surgery. However, in some embodiments, the attachment point 301 can be configured to accept a corresponding component that is permanently fixed to the upper bone anchor 300. Alternatively, the upper bone anchor 300 may be configured to detach at any point to achieve a similar detachable function.

In FIG. 20B is a third exemplary detachable lower bone anchor 310 configured to removably connect to the attachment point 301 on upper bone anchor 300. The lower bone anchor 310 is preferentially comprised, in part or completely, of a repeating unit cell lattice structure. The lower tip 312 of the lower bone anchor 310 may comprise a solid material or a solid surface in some embodiments to facilitate tissue distraction during implantation.

The lower bone anchor 310 can be configured to attach to a specific type of attachment point 301. In some embodiments, the lower bone anchor 310 can be threaded on its circumferential surface. If the lower bone anchor 310 is threaded, the thread pitch can be substantially similar to the thread pitch on the upper bone anchor (if also threaded), different or progressively different.

In FIG. 18C is an alternative third exemplary detachable lower bone anchor 310A configured to removably connect to attachment point 301 on upper bone anchor 300. The lower bone anchor 310A is preferentially comprised, in part or completely, of a repeating unit cell lattice structure. The lower tip 312A of the lower bone anchor 310A may comprise a solid material in some embodiments to facilitate tissue distraction during implantation. The solid or substantially solid material, when used on the lower tip 312A, does not need to extend significantly below the surface of the lower tip 312A and can be merely a solid or substantially solid shell or coating. In some embodiments, solid or substantially solid can refer to an area of the bone anchor that is configured to facilitate tissue distraction during implantation rather than a specific volumetric density. In some embodiments, solid or substantially solid can refer to a substantially smooth or relatively smooth, compared to the roughness of other areas of the bone screw or bone anchor, outer layer configured to facilitate tissue distraction during implantation

The lower bone anchor 310A can be configured to attach to a type of attachment point 301. For example, if the attachment point 301 is a threaded cylindrical protrusion, the lower bone anchor 310A could have a corresponding threaded cylindrical opening 311A for a threaded fit. In some embodiments, the lower bone anchor 310A can be threaded on its circumferential surface. If the lower bone anchor 310A is threaded, the thread pitch can be substantially similar to the thread pitch on the upper bone anchor (if also threaded), can be different or can progressively change in thread pitch.

In FIG. 20A is a top sectioned view of a fourth exemplary embodiment of a lower bone anchor 410. The lower bone anchor 410 can have a cylindrical cross section in some embodiments to make the implant easier to rotate about its axis. In FIG. 20B is a top sectioned view of a fifth exemplary embodiment of a lower bone anchor 510. The lower bone anchor 510 can have a triangular cross section to make the implant more difficult to rotate about its axis. The cross-sectional shape of the lower bone anchors 410 & 510 can optionally be used in any of the previously disclosed lower bone anchors 110, 210 & 310 or upper bone anchors 100, 200 & 300.

Any of the bone anchors described herein do not need to have a uniform diameter, shape or cross-sectional area throughout their length. The use of irregularities along the length of a bone anchor with respect to diameter, shape or cross-sectional area could enhance the holding power of the bone anchor in a substrate over a perfectly uniform bone anchor.

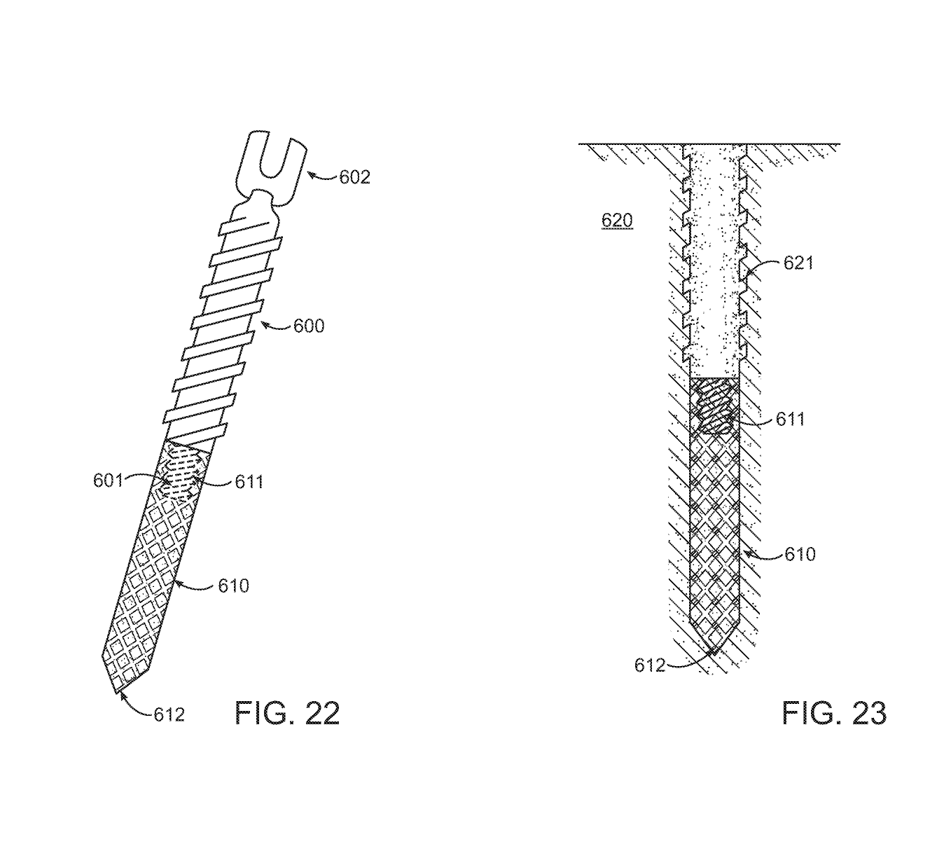

In FIG. 22 is a side view of a bone screw assembly 602 comprising an upper bone anchor 600 attached to a lower bone anchor 610. In this example, the upper bone anchor 600 uses a cylindrical protrusion 611 that engages an attachment point 601 in the lower bone anchor 610. The cylindrical protrusion 611 and the attachment point 601 are generally located about the longitudinal axis of the bone screw assembly 602 but are shown in this side view for additional clarity. The lower bone anchor 610 may be comprised in part or entirely of a lattice structure. The upper bone anchor 600 may be comprised in part or entirely of a lattice structure in some embodiments.

In FIG. 23 is a side sectioned view of a bone screw assembly after implantation and after the upper bone anchor has been removed. The bone screw assembly can be implanted in an opening 621 within a segment of bone 620. The opening 621 can be created through multiple processes, such as, using a drilling process, awling or by the bone screw assembly itself during implantation. In some embodiments, the lower bone anchor 610 is configured to attach, resorb or osteointegrate with the bone 620 and the upper bone anchor 600 is configured to substantially resist bone in-growth. In embodiments that allow bone attachment or in-growth into the lower bone anchor 610, the lower bone anchor 610 will become much more difficult to remove over time than the upper bone anchor. In FIG. 23, the upper bone anchor has been removed, leaving the lower bone anchor 610 within the opening 621 in the bone 620.

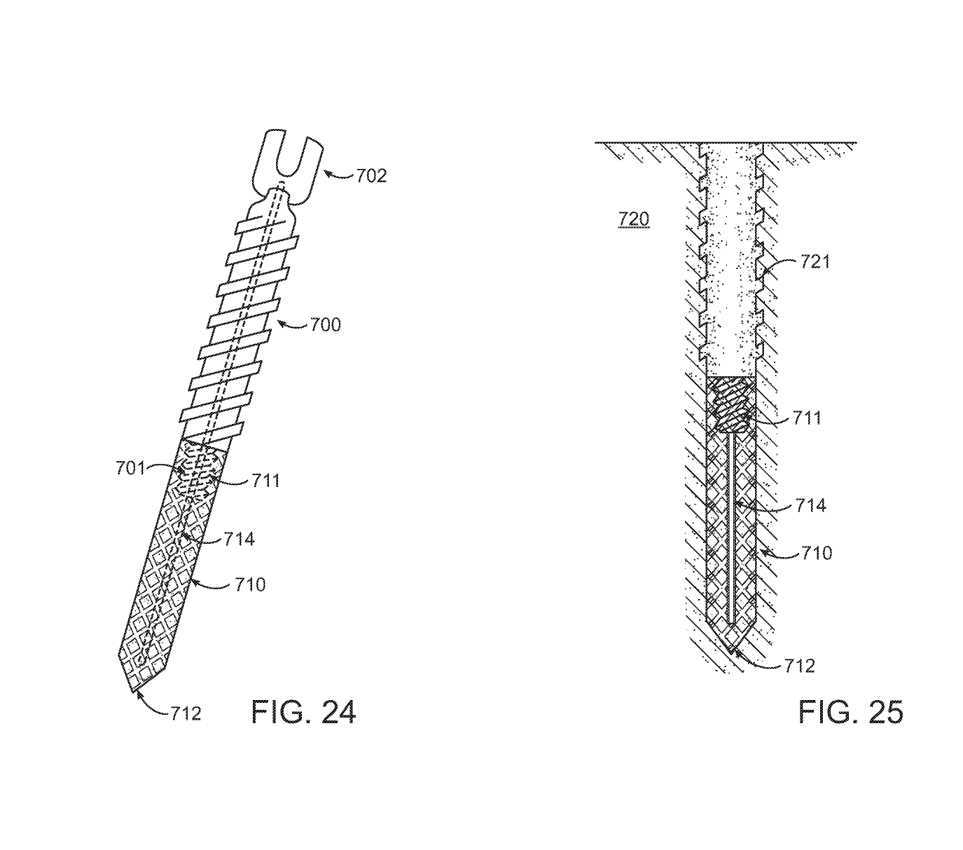

In FIG. 24 is a side view of a bone screw assembly 702 comprising an upper bone anchor 700 attached to a lower bone anchor 710. In this example, the upper bone anchor 700 uses a cylindrical protrusion 711 that engages an attachment point 701 in the lower bone anchor 710. The cylindrical protrusion 711 and the attachment point 701 are generally located about the longitudinal axis of the bone screw assembly 702 and are shown in dashed lines because they are hidden in this view. The lower bone anchor 710 may comprise, in part or entirely, of a lattice structure. The upper bone anchor 700 may comprise, in part or entirely, of a lattice structure in some embodiments. In some embodiments, the bone screw assembly 702 further comprises a cannula 714 or opening. The cannula 714 can be configured along the axial center of the bone screw assembly 702 in some embodiments or preferentially placed along a surface of the bone screw assembly 702 in some embodiments. The cannula 714 can terminate within the lower bone anchor 710 before the tip 712.

In FIG. 25 is a side sectioned view of a bone screw assembly after implantation and after the upper bone anchor has been removed. The bone screw assembly can be implanted in an opening 721 within a segment of bone 720. The opening 721 can be created through multiple processes, such as, using a drilling process, awling or by the bone screw assembly itself during implantation. In some embodiments, the lower bone anchor 710 is configured to attach, allow bone ingrowth, resorb or osteointegrate with the bone 720 and the upper bone anchor 700 is configured to substantially resist bone in-growth. In embodiments that allow bone attachment or in-growth into the lower bone anchor 710, the lower bone anchor 710 will become much more difficult to remove over time than the upper bone anchor. In FIG. 25, the upper bone anchor has been removed, leaving the lower bone anchor 710 within the opening 721 in the bone 720.

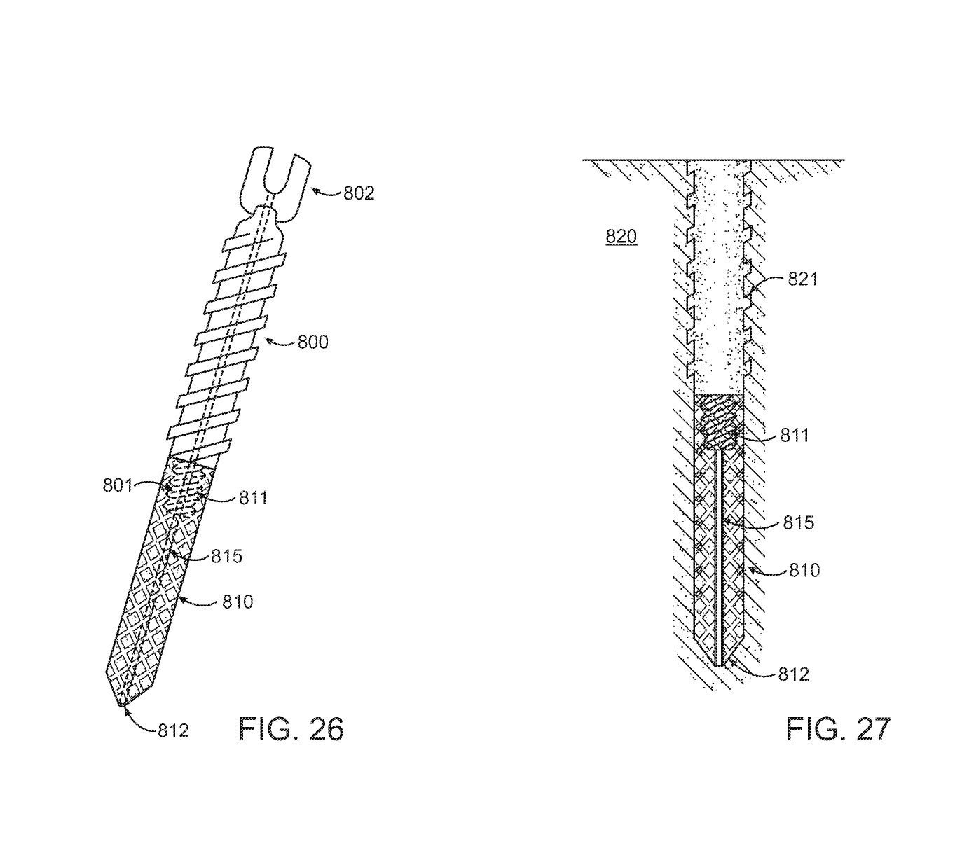

In FIG. 26 is a side view of a bone screw assembly 802 comprising an upper bone anchor 800 attached to a lower bone anchor 810. In this example, the upper bone anchor 800 uses a cylindrical protrusion 811 that engages an attachment point 801 in the lower bone anchor 810. The cylindrical protrusion 811 and the attachment point 801 are generally located about the longitudinal axis of the bone screw assembly 802 and are shown in dashed lines because they are hidden in this view. The lower bone anchor 810 may comprise, in part or entirely, of a lattice structure. The upper bone anchor 800 may comprise, in part or entirely, of a lattice structure in some embodiments. In some embodiments, the bone screw assembly 802 further comprises a cannula 815 or opening. The cannula 815 can be configured along the axial center of the bone screw assembly 802 in some embodiments or preferentially placed along a surface of the bone screw assembly 802 in some embodiments. The cannula 815 can terminate through the tip 812 of the lower bone anchor 810.

In FIG. 27 is a side sectioned view of a bone screw assembly after implantation and after the upper bone anchor has been removed. The bone screw assembly can be implanted in an opening 821 within a segment of bone 820. The opening 821 can be created through multiple processes, such as, using a drilling process, awling or by the bone screw assembly itself during implantation. In some embodiments, the lower bone anchor 810 is configured to attach, allow bone ingrowth, resorb or osteointegrate with the bone 820 and the upper bone anchor 800 is configured to substantially resist bone in-growth. In embodiments that allow bone attachment or in-growth into the lower bone anchor 810, the lower bone anchor 810 will become much more difficult to remove over time than the upper bone anchor. In FIG. 27, the upper bone anchor has been removed, leaving the lower bone anchor 810 within the opening 821 in the bone 820.

In some embodiments, the upper bone anchor or lower bone anchor can comprise a lattice with anisotropic properties. In some embodiments, the scaffold may be anisotropic in the rotation direction. In other embodiments, the scaffold may be anisotropic in one or more shear directions. In some embodiments, the lattice comprises an anisotropy where the lattice is elongated in substantially the same direction as the elongate direction of the upper bone anchor or the lower bone anchor. A bone anchor comprising a lattice that is elongated in the same direction as the elongate direction of the bone anchor could provide enhanced strength characteristics, such as additional strength when the bone anchor is hammered or struck along its elongate direction.

Some embodiments can include internal and external indicators to convey certain types of information to a surgeon or medical professional. Some embodiments use indicators on the bone anchor to indicate the direction of anisotropy. Indicators can include writings or markings on the implant or modifications to the lattice structure to achieve a predetermined pattern, such as through including x-ray markers as previously disclosed. In some embodiments, x-ray markers indicate the direction of anisotropy through increased relative radiolucency or opacity. In some embodiments, x-ray markers indicate the direction of anisotropy through increased relative radiolucency or opacity. In some embodiments, x-ray markers indicate, through increased relative radiolucency or opacity, the direction of other structures or inclusions in the device.

Some embodiments comprise a lattice that is anisotropic in the rotational direction of the bone anchor, where the anisotropy is perpendicular to the radial arm from the central axis. When configured with a rotational anisotropy, the lattice can be configured to be higher stiffness in the direction of rotation of the screw. The rotational anisotropy can be accomplished through layering unit cells of varying properties radially from the axial center of the bone anchor. The rotational anisotropy could also be accomplished adjusting normal unit cells to be variably anisotropic within the normal layers moving away from the axial center of the bone anchor (e.g., by using increasingly wider unit cells further from the axial center of the bone anchor).

In some embodiments, the lattice structure is supported in part by a frame of beam structures larger than those of the lattice structural members either internal or external to the lattice body.

In some embodiments, the anchor will be comprised of one body. In some embodiments, the bone screws or anchors may be constructed as a single component or part. Some embodiments may be manufactured as a single part with a selective separation area configured to remain connected during insertion and configured to separate during removal or revision surgery. The selective separation area could comprise a unidirectional portion with a step or similar structure. Some embodiments are strong in localized compression and weak in localized extension around the selective separation area.

In some embodiments, a bone anchor is comprised of an upper bone anchor removably fixed to a lower bone anchor. When the upper and lower bone anchors are fixed together, they act as a unitary device to facilitate implantation. In bone anchors designed to be removable, for example during a revision surgery, the upper bone anchor can be largely of solid construction and the lower bone anchor can be of largely lattice construction. After implantation, the lattice lower bone anchor will tend to osseointegrate and/or resorb with the surrounding bone through the process of bone ingrowth. The upper bone anchor will attach to the surrounding bone, but if solid, bone ingrowth will not significantly occur. The upper bone anchor may optionally include a coating to resist bone attachment. The implanted bone anchor could then be partially removed by decoupling the upper bone anchor from the lower bone anchor and using conventional means to remove the upper bone anchor.

The bone anchors disclosed herein can be used for multiple types of medical implants, including but not limited to, the attachment of bone plates, interbodies, wedges and teeth.

What has been described is a bone anchor for temporary or permanent attachment to bone. In this disclosure, there are shown and described only exemplary embodiments of the invention, but, as aforementioned, it is to be understood that the invention is capable of use in various other combinations and environments and is capable of changes or modifications within the scope of the inventive concept as expressed herein.

What is claimed is:

1. A bone anchor for medical implants, comprising:
a first elongate segment and a second elongate segment;
wherein:
the first elongate segment comprises a cylindrical cross section with a helix shaped protrusion fixed along a circumferential surface;
the first elongate segment further comprises a first attachment area and the second elongate segment further comprises a second attachment area, wherein the first attachment area is configured to selectively attach to the second attachment area; and
the second elongate segment comprises a metallic lattice.

2. The bone anchor of claim 1, wherein the second elongate segment further comprises a cylindrical cross section with two ends; a first end housing the second attachment area and a second end comprising a volume with a volumetric density of more than 85%.

3. The bone anchor of claim 1, wherein the second elongate segment further comprises a cylindrical cross section with two ends; a first end housing the second attachment area and a second end configured to enhance tissue distraction.

4. The bone anchor of claim 1, wherein the first attachment area comprises a cylindrical extension and the second attachment area comprises a cylindrical opening configured to accept the first attachment area.

5. The bone anchor of claim 4, wherein the first attachment area fits within the second attachment area with an interference fit.

6. The bone anchor of claim 1, wherein the first attachment area comprises a threaded cylindrical extension and the second attachment area comprises a threaded opening configured to accept the first attachment area.

7. The bone anchor of claim 1, wherein the first elongate segment has a volumetric density of about 100%.

8. The bone anchor of claim 1, wherein the first elongate segment has a volumetric density of greater than 85%.

9. The bone anchor of claim 1, wherein the first elongate segment further comprises a surface treatment inhibiting the attachment of bone growth.

10. The bone anchor of claim 1, wherein the second elongate segment further comprises a surface treatment enhancing the ingrowth of bone.

11. The bone anchor of claim 10, wherein the first elongate segment further comprises a surface treatment inhibiting the attachment of bone growth.

12. The bone anchor of claim 1, wherein the second elongate segment further comprises a cylindrical cross section.

13. The bone anchor of claim 12, wherein the second elongate segment further comprises a circumferential surface with a threaded pattern.

14. The bone anchor of claim 1, wherein the second elongate segment further comprises a triangular cross section.

15. The bone anchor of claim 1, wherein the first elongate segment is substantially nonresorbable and the second elongate segment is resorbable.

16. The bone anchor of claim 1, wherein the first elongate segment is substantially nonresorbable and the second elongate segment is configured for bone ingrowth.

17. The bone anchor of claim 1, wherein the first attachment area comprises a threaded cylindrical opening and the second attachment area comprise a threaded cylindrical extension configured to thread into the first attachment area.

18. The bone anchor of claim 1, wherein the first segment further comprises an irregular cylindrical cross section.

19. The bone anchor of claim 18, wherein the second segment further comprises an irregular cross section.

20. The bone anchor of claim 6, wherein the second attachment area further comprises an outer layer of material with a volumetric density of at least 85% and at least 0.5 mm.
